(19) United States
(12) Patent Application Publication
(10) Pub. No.: US 2012/0040953 A1
(43) Pub. Date: Feb. 16, 2012

(54) Compounds for the Treatment of Metabolic Disorders

(75) Inventors: Oscar Barba, Oxford (GB); Susan Helen Davis, Oxford (GB); Matthew Colin Thor Fyfe, Oxford (GB); Revathy Perpetua Jeevaratnam, Oxford (GB); Karen Lesley Schofield, Oxford (GB); Thomas Staroske, Oxford (GB); Alan John William Stewart, Oxford (GB); Simon Andrew Swain, Oxford (GB); David Matthew Withall, Oxford (GB)

(73) Assignee: PROSIDION LIMITED, Oxford (GB)

(21) Appl. No.: 13/255,536

(22) PCT Filed: Mar. 12, 2010
(86) PCT No.: PCT/GB2010/050442

§ 371 Date: Nov. 2, 2011

(30) Foreign Application Priority Data
Mar. 12, 2009 (GB) 0904287.0

(51) Int. Cl. A61K 31/506; A61K 31/4545; C07D 401/14; A61P 3/06; A61P 3/00; A61P 3/04; C07D 413/14; A61K 31/501; A61P 3/10

(52) U.S. Cl. 514/210.18; 514/252.03; 514/275; 514/316; 514/318; 544/238; 544/331; 546/187; 546/194

(57) ABSTRACT

The present invention is directed to therapeutic compounds which have activity as agonists of GPR119 and are useful for the treatment of metabolic disorders including type II diabetes.

### BACKGROUND OF THE INVENTION

The present invention is directed to therapeutic compounds useful for the treatment of metabolic disorders including type II diabetes. In particular, the present invention is directed to compounds which have activity as agonists of GPR119.

Drugs aimed at the pathophysiology associated with non-insulin dependent type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) places people at high risk of coronary artery disease, and is characterized by a cluster of risk factors including central obesity (excessive fat tissue in the abdominal region), glucose intolerance, high triglycerides and low HDL cholesterol, and high blood pressure. Myocardial ischemia and microvascular disease is an established morbidity associated with untreated or poorly controlled metabolic syndrome.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

There is a continuing need for novel antidiabetic agents, particularly ones that are well tolerated with few adverse effects and in particular for agents which are weight neutral or preferably cause weight loss.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of diabetes.

GPR119 agonists have been shown to stimulate the release of GLP-1 from the GI tract. In doing so, GPR119 agonists (1) enhance glucose-dependent insulin release from the pancreas leading to improvements in oral glucose tolerance; (2) attenuate disease progression by increasing β-cell cAMP concentrations; and (3) induce weight loss possibly through GLP-1's ability to reduce food intake.

International Patent Applications WO2005/061489, WO2006/070208, WO2006/067532, WO2006/067531, WO2007/003960, WO2007/003961, WO2007/003962, WO2007/003964, WO2007/116229, WO2007/116230, WO2007/138362, WO2008/081204, WO2008/081205, WO2008/081206, WO2008/081207, WO2008/081208, WO2009/050522, WO2009/050971, WO2010/004343, WO2010/004344, WO2010/004345, WO2010/004347 and WO2010/00166 disclose GPR119 receptor agonists.

Dipeptidyl peptidase IV (DPP-IV) is a ubiquitous, yet highly specific, serine protease that cleaves N-terminal dipeptides from polypeptides with L-proline or L-alanine at the penultimate position. Studies with DPP-IV inhibitors show the principle role of DPP-IV is in the inactivation GLP-1. By extending the duration of action of GLP-1, insulin secretion is stimulated, glucagon release inhibited, and gastric emptying slowed. DPP-IV inhibitors are of use for the treatment of type II diabetes, examples of DPP-1V inhibitors include vildagliptin, sitagliptin, alogliptin and saxagliptin.

The possibility of using a combination of a GPR119 agonist and a DPP-IV inhibitor has been suggested, however this requires the administration of two separately formulated products to the patient or the co-formulation of two active ingredients with the inherent problems of achieving compatibility in the physicochemical, pharmacokinetic and pharmacodynamic properties of the two active ingredients. International Patent Application WO2009/034388, published after the priority date of the present application, discloses compounds having dual activity as agonists of GPR119 and inhibitors of DPP-IV.

The compounds of the invention may also have dual activity as agonists of GPR119 and inhibitors of DPP-IV.

### SUMMARY OF THE INVENTION

The present invention is directed to compounds which have activity as agonists of GPR119 and may also be inhibitors of DPP-IV and are useful for the treatment of metabolic disorders including type II diabetes.

### DETAILED DESCRIPTION OF THE INVENTION

The present invention provides compounds of formula (I) and pharmaceutically acceptable salts thereof:

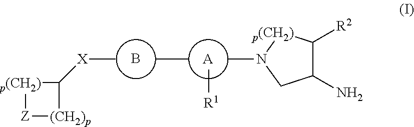

wherein p is 1 or 2;

when p is 2, Z is N—C(O)OR⁴, N—C(O)NR⁴R⁵ or N-heteroaryl which may optionally be substituted by one or two groups selected from C₁₋₄ alkyl, C₃₋₆ cycloalkyl optionally substituted by C₁₋₄ alkyl, C₁₋₄ alkoxy, C₁₋₄ haloalkyl and halogen;

when p is 1, Z can also be —N—CH₂-phenyl wherein the phenyl is optionally substituted by 1 or 2 groups independently selected from C₁₋₄ alkyl, C₁₋₄ haloalkyl and halo;

A is a para-substituted phenyl or a para-substituted 6-membered heteroaryl ring containing one or two nitrogen atoms;

B is a 5-membered heteroaryl ring containing one of more heteroatoms selected from N, O and S or, a para-substituted 6-membered heteroaryl ring containing one or two nitrogens;

when B is a 5-membered heteroaryl ring X is —O—CR⁶H— or —CR⁷H—O—CR⁶H—; and when B is a 6-membered heteroaryl ring X is —O— or CR⁶H—O—;

R¹ is hydrogen, halo, cyano, C₁₋₄ alkyl or C₁₋₄ haloalkyl;

q is 1 or 2;

R² is

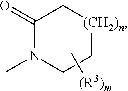

phenyl optionally substituted by one or more halo groups, or pyridyl optionally substituted by one or more halo or methyl groups;

R³ is independently halo or methyl;

n is 0 or 1;

m is 0, 1 or 2;

R⁴ is C₂₋₆ alkyl or C₃₋₆ cycloalkyl wherein the cycloalkyl is optionally substituted by C₁₋₄alkyl;

R⁵ is hydrogen or C₁₋₄ alkyl; and

R⁶ and R⁷ are independently hydrogen or C₁₋₂ alkyl.

In a preferred embodiment the compounds of the invention have the stereochemistry as defined in formula (Ia), such compounds demonstrate DPP-IV inhibitory activity:

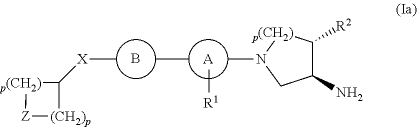

In one of embodiment of the invention each p is independently 1 or 2, i.e. forming a 4-, 5- or 6-membered ring. In another embodiment of the invention each p is the same, i.e. forming a 4- or 6-membered ring. In the compounds of the invention p is preferably 2.

In one embodiment of the invention Z is N—C(O)OR⁴.

In a further embodiment of the invention Z is N-heteroaryl which may optionally be substituted by one or two groups selected from C₁₋₄ alkyl, C₃₋₆ cycloalkyl optionally substituted by C₁₋₄alkyl, C₁₋₄ alkoxy, C₁₋₄ haloalkyl and halogen.

When Z is N-heteroaryl preferred heteroaryl groups include oxadiazole and pyrimidine.

A is preferably phenyl, pyridyl or pyrimidinyl.

R¹ is preferably hydrogen.

In one embodiment of the invention B is a 5-membered heteroaryl ring, in another B is a 6-membered heteroaryl ring.

When B is a 5-membered heteroaryl ring X is preferably —O—CR⁶H—; and when B is a 6-membered heteroaryl ring X is preferably or CR⁶H—O—.

R² is preferably phenyl or pyridyl, more preferably phenyl, and even more preferably substituted phenyl.

When R² is phenyl substituted by one or more halo groups it is preferably substituted by 1 to 3 halo groups, the halo groups are preferably fluoro or chloro, more preferably fluoro.

When R² is pyridyl it is preferably 2-pyridyl.

When R² is substituted pyridyl it is preferably substituted by 1 to 3 halo or methyl groups, more preferably 1 or 2 methyl groups.

n is preferably 1.

R⁴ is preferably C₂₋₆ alkyl.

R⁶ and R⁷ are independently preferably hydrogen or methyl.

A group of compounds which may be mentioned are those of formula (Ib) and pharmaceutically acceptable salts thereof:

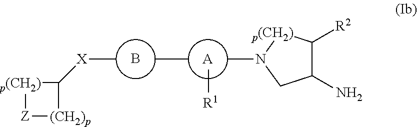

wherein p is 1 or 2;

when p is 2, Z is N—C(O)OR⁴, N—C(O)NR⁴R⁵ or N-heteroaryl which may optionally be substituted by one or two groups selected from C₁₋₄ alkyl, C₁₋₄ alkoxy, C₁₋₄ haloalkyl and halogen;

when p is 1, Z is —N—CH₂-phenyl wherein the phenyl is optionally substituted by 1 or 2 groups independently selected from C₁₋₄alkyl, C₁₋₄haloalkyl and halo;

A is a para-substituted phenyl or a para-substituted 6-membered heteroaryl ring containing one or two nitrogen atoms;

B is a 5-membered heteroaryl ring containing one of more heteroatoms selected from N, O and S or, a para-substituted 6-membered heteroaryl ring containing one or two nitrogens;

when B is a 5-membered heteroaryl ring X is —O—CR⁶H— and when B is a 6-membered heteroaryl ring X is —O— or CR⁶H—O—;

R¹ is hydrogen, halo, cyano, C₁₋₄alkyl or C₁₋₄haloalkyl;

q is 1 or 2;

R² is

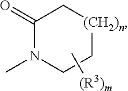

or phenyl optionally substituted by one or more halo groups;

R³ is independently halo or methyl;

n is 0 or 1;

m is 0, 1 or 2;

R⁴ is C₂₋₆ alkyl;

R⁵ is C₁₋₄alkyl; and

R⁶ is hydrogen or C₁₋₂alkyl.

In a preferred embodiment of the compounds formula (Ib) they have the stereochemistry as defined in formula (Ia).

While the preferred groups for each variable have generally been listed above separately for each variable, preferred compounds of this invention include those in which several or each variable in formula (I) is selected from the preferred groups for each variable. Therefore, this invention is intended to include all combinations of preferred listed groups.

The molecular weight of the compounds of the invention is preferably less than 800, more preferably less than 600.

As used herein, unless stated otherwise, “alkyl” means carbon chains which may be linear or branched. Examples of alkyl groups include ethyl, propyl, isopropyl, butyl, sec- and tert-butyl.

The term “heteroaryl” rings means 5- or 6-membered N-containing heteroaryl rings containing up to 2 additional heteroatoms selected from N, O and S. Examples of such heteroaryl rings are pyrrolyl, pyrazolyl, imidazolyl, oxazolyl, isoxazolyl, thiazolyl, isothiazolyl, triazolyl, oxadiazolyl, thiadiazolyl, pyridinyl, pyridazinyl, pyrimidinyl, pyrazinyl and triazinyl.

Reference to para substitution in relation to rings A and B refers to the positions of the group —B— and the N-containing heterocycle on ring A and groups —X— and -A- on ring B.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The present invention includes all stereoisomers of the compounds of the invention and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of the invention exists, the present invention includes any possible tautomers and pharmaceutically acceptable salts thereof, and mixtures thereof, except where specifically drawn or stated otherwise.

When the compound of the invention and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like

Since the compounds of the invention are intended for pharmaceutical use they are preferably provided in substantially pure form, for example at least 60% pure, more suitably at least 75% pure, especially at least 98% pure (% are on a weight for weight basis).

The compounds of formula (I) can be prepared as described below, wherein R¹, R², R³, R⁴, R⁵, R⁶, A, B, X, Z, m, n, p, q are as defined for formula (I). PG is a protecting group, Hal is halogen and E is either halogen or triflate.

Compounds of formula (I) can be prepared as outlined in Scheme 1. Compounds of formula (IV) can be prepared by SN_{Ar} displacement of suitable haloaromatic compounds of formula (II) with amines of formula (III) under standard conditions, for example, DBU and DMSO at 120° C. Alternatively, compounds of formula (IV) can be prepared by reaction of suitable haloaromatic compounds of formula (II) with amines of formula (III) under Buchwald-Hartwig conditions, such as, Pd₂(dba)₃ and BINAP in a suitable solvent, such as toluene at 110° C. Deprotection of the amine functionality, using standard conditions well known to those with skill in the art, affords compounds of formula (I) as described above.

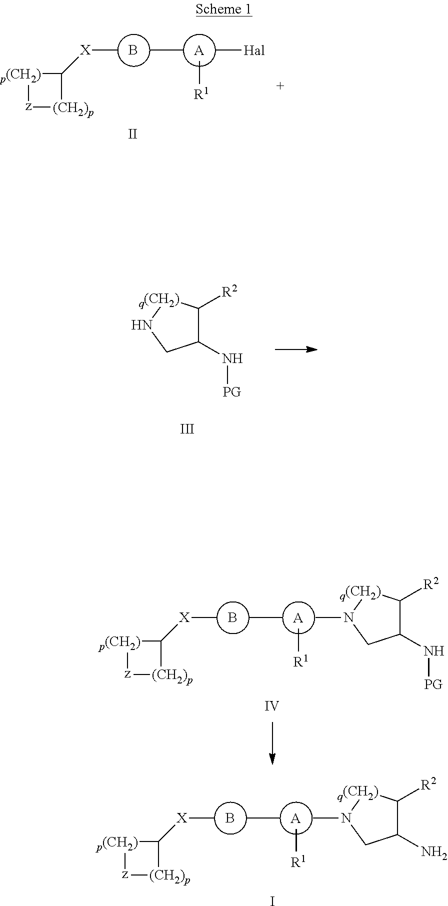

Building blocks of formula (II), where B is a para-substituted 6-membered heteroaryl ring containing one or two nitrogens and X is —O— or CR⁶H—O—, can be prepared as outlined in Scheme 2. Aryl halides of formula (V) can be treated with boronates of formula (VI) under standard Suzuki conditions, for example, [1,1-bis(diphenylphosphino)ferrocene]dichloropalladium in a suitable solvent such as DMF/water at 80° C.

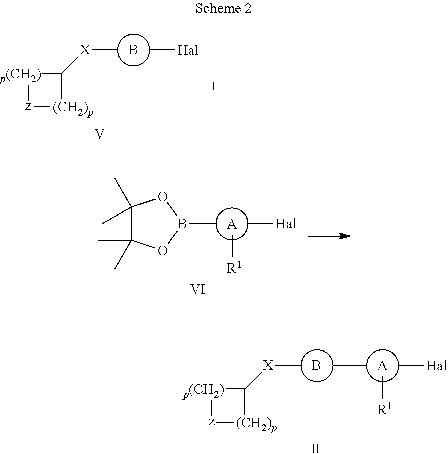

Alternatively, building blocks of formula (II), where B is a para-substituted 6-membered heteroaryl ring containing one or two nitrogens and X is —O— or CR⁶H—O—, can be prepared as outlined in Scheme 3. Aryl boronates of formula (VII) can be prepared by reaction of aryl halide of formula (V) and bis(pinacolato)diboron in the presence of a suitable catalyst, such as [1,1-bis(diphenylphosphino)ferrocene]dichloropalladium in a suitable solvent such as 1,4-dioxane at 110° C. Building blocks of formula (II) can be prepared by reaction of boronates of formula (VII) with aryl halides or aryl triflates of formula (VIII) under standard Suzuki conditions, for example, [1,1-bis(diphenylphosphino)ferrocene]dichloropalladium in a suitable solvent such as DMF/water at 80° C.

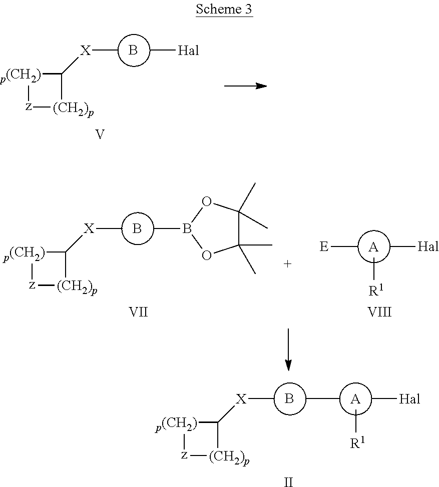

Building blocks of formula (V) where B is a para-substituted 6-membered heteroaryl ring containing one or two nitrogens and X is —O— or CR⁶H—O—, can be prepared as outlined in Scheme 4. Alcohols of formula (IX) can be treated with hydroxyaryls of formula (X) under standard Mitsonobu conditions, for example, using azodicarboxylic dipiperidide and tributylphosphine in a suitable solvent such as toluene.

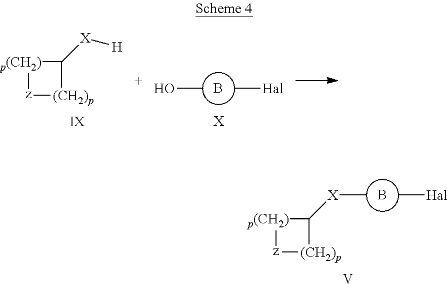

Alternatively, building blocks of formula (V) where B is a para-substituted 6-membered heteroaryl ring containing one or two nitrogens and X is —O— or CR⁶H—O—, can be prepared as outlined in Scheme 5. Alcohols of formula (IX) can be treated with a suitable dihaloaryl compound of formula (XI) under standard SN_{Ar} conditions, such as DBU and DMSO at 120° C.

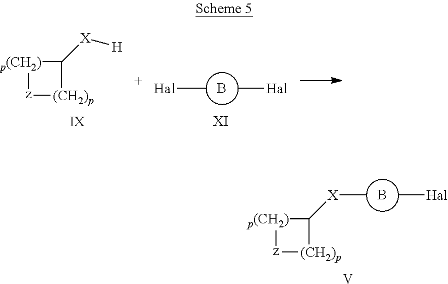

Building blocks of formula (II) where B is a 1,2,4-oxadiazol-5-yl and X is —O—CR⁶H— can be prepared as outlined in Scheme 6. Amidoxime of formula (XII) can be prepared by reaction of nitrile of formula (XIII) and hydroxylamine hydrochloride in the presence of a suitable base such as K₂CO₃ in a suitable solvent such as ethanol/water at 78° C. Building blocks of formula (II) as described above can be prepared by reaction of amidoxime of formula (XII) with acid of formula (XIV) under standard conditions, such as isobutyl chloroformate and triethylamine, in a suitable solvent such as DMF.

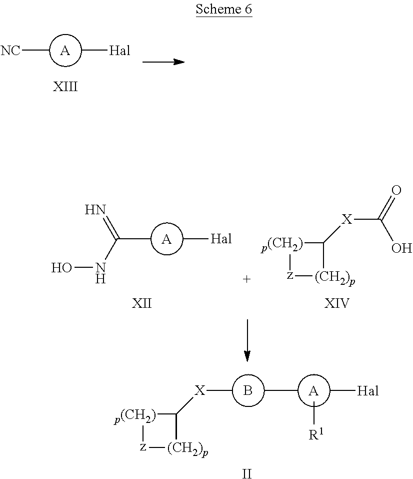

Building blocks of formula (II) where B is a 1,2,4-oxadiazol-3-yl and X is —O—CR⁶H— can be prepared as outlined in Scheme 7. Amidoximes of formula (XV) can be prepared by reaction of nitrile of formula (XVI) and hydroxylamine hydrochloride in the presence of a suitable base such as K₂CO₃ in a suitable solvent such as ethanol/water at 78° C. Building blocks of formula (II) as described above can be prepared by reaction of amidoxime of formula (XV) with acid of formula (XVII) under standard conditions, such as isobutyl chloroformate and triethylamine, in a suitable solvent such as DMF.

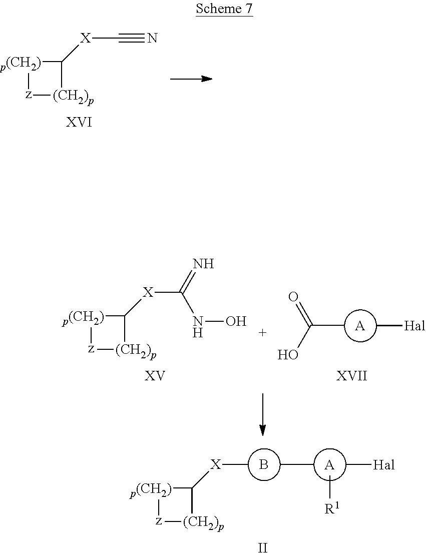

Building blocks of formula (II) where B is a thiazol-2-yl and X is —O—CR⁶H— can be prepared as outlined in Scheme 8. Primary amides of formula (XVIII) can be prepared by reaction of acids of formula (XIV) with ammonia in 1,4-dioxane solution under standard amide coupling conditions, for example, HOBT and EDCI, in a suitable solvent, such as DCM. Thioamides of formula (XIX) can be prepared by reaction of primary amides of formula (XVIII) under standard conditions, for example using Lawesson's reagent in a suitable solvent such as toluene at reflux. Building blocks of formula (II) as described above can be prepared by reaction of bromoketones of formula (XX) with thioamide of formula (XIX) under standard Hantzsch conditions, for example ethanol at room temperature.

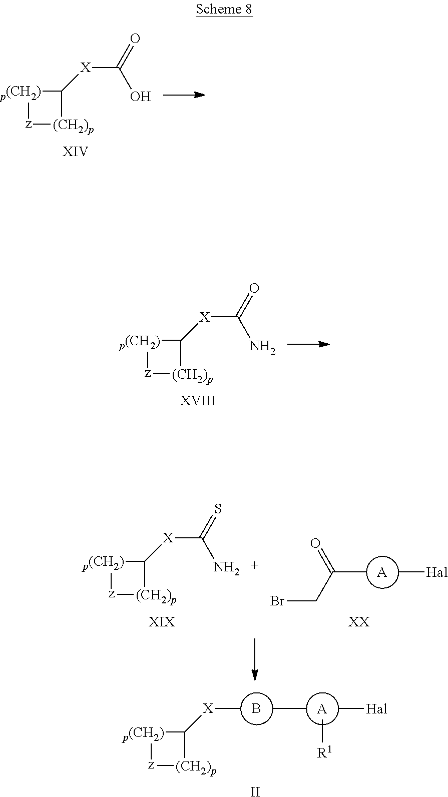

Building blocks of formula (II) where B is a thiazol-5-yl and X is —O—CR⁶H— can be prepared as outlined in Scheme 9. Primary amides of formula (XXI) can be prepared by reaction of acids of formula (XVII) with ammonia in 1,4-dioxane solution under standard amide coupling conditions, for example, HOBT and EDCI, in a suitable solvent, such as DCM. Thioamides of formula (XXII) can be prepared by reaction of primary amides of formula (XVIII) under standard conditions, for example using Lawesson's reagent in a suitable solvent such as toluene at reflux. Building blocks of formula (II) as described above can be prepared by reaction of chloroketones of formula (XXIII) with thioamide of formula (XXII) under standard Hantzsch conditions, for example ethanol at room temperature.

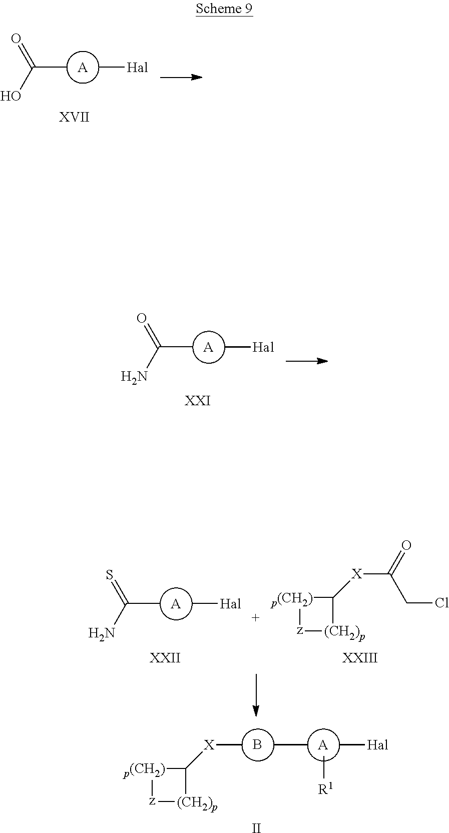

Building blocks of formula (XVI) where X is —O—CR⁶H— can be prepared as outlined in Scheme 10. Alcohols of formula (XXIV) can be treated with bromides of formula (XXV) under standard conditions, for example, NaH in a suitable solvent, such as THF at 0° C.

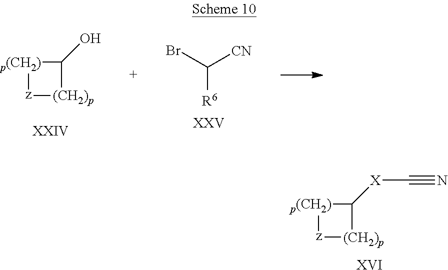

Building blocks of formula (XX) can be prepared as outlined in Scheme 11. Ketones of formula (XXVI) can be treated with trimethylphenylammonium tribromide in a suitable solvent, such as THF.

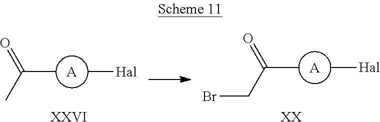

Building blocks of formula (XXIII) where X is —O—CR⁶H— can be prepared as outlined in Scheme 12. Alcohols of formula (XXIV) can be treated with 1,3-dichloroacetone in the presence of a suitable base, such as K₂CO₃, in a suitable solvent such as DMF.

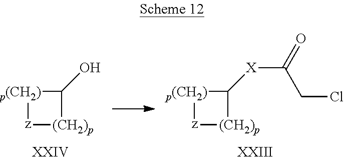

Examples and syntheses of building blocks of formula (III) have been described elsewhere: Benbow et.al., WO2007/148185; Brackes et.al., Bioorg. Med. Chem. Lett., 2007, 17 2005-2012; Pei et.al., J. Med. Chem., 2007, 50 (8), 1983-1987; Cox et.al., Bioorg. Med. Chem. Lett., 2007, 17 4579-4583; Wright et.al., Bioorg. Med. Chem. Lett., 2007, 17 5638-5642.

The synthesis of building blocks of formula (IX) where p is 2 and X is —O— or CR⁶H—O— have been described elsewhere: Fang et. al., WO2008/070692; Alper et. al., WO2008/097428; Wacker et.al., WO2009/012275.

The synthesis of building blocks of formula (IX) where p is 1 and X is —O— or CR⁶H—O— have been described elsewhere: Arnould et.al., WO2007/091046; Evans et.al., WO2008/079028.

The synthesis of building blocks of formula (XIV) where X is —O—CR⁶H— have been described elsewhere: Bertram et.al., WO2007/116229.

Other compounds of formula (I) may be prepared by methods analogous to those described above or by methods known per se. Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel syntheses using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition.

The processes for the production of the compounds of formula (I) and intermediates thereto as described above are also included as further aspects of the present invention.

Any novel intermediates as defined in the Schemes above or in the Examples, are also included within the scope of the invention. Therefore according to a further aspect of the invention there is provided a compound of any one of formulae (II), (IV), (V), (XIV), (XV), (XVI), (XVIII), (XIX) and (XXIII) as defined above. The preferred groups for variables recited above in relation to the compounds of formula (I) also apply to the intermediates compounds.

As indicated above the compounds of the invention are useful as GPR119 agonists, e.g. for the treatment and/or prophylaxis of diabetes. For such use the compounds of the invention will generally be administered in the form of a pharmaceutical composition.

The compounds of the invention may also be useful as dual GPR119 agonists/DPP-IV inhibitors, e.g. for the treatment and/or prophylaxis of diabetes. For such use the compounds of the invention will generally be administered in the form of a pharmaceutical composition.

The invention also provides a compound of the invention, or a pharmaceutically acceptable salt thereof, for use as a pharmaceutical.

The invention also provides a pharmaceutical composition comprising a compound of the invention, in combination with a pharmaceutically acceptable carrier.

Preferably the composition is comprised of a pharmaceutically acceptable carrier and a non-toxic therapeutically effective amount of a compound of the invention, or a pharmaceutically acceptable salt thereof.

Moreover, the invention also provides a pharmaceutical composition for the treatment of disease by modulating GPR119 and optionally DPP-IV, resulting in the prophylactic or therapeutic treatment of diabetes, comprising a pharmaceutically acceptable carrier and a non-toxic therapeutically effective amount of compound of the invention, or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of the invention, or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of the invention, or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of the invention, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of the invention, or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of the invention, or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of the invention may be used in the treatment of diseases or conditions in which GPR119 and optionally DPP-IV play a role.

Thus the invention also provides a method for the treatment of a disease or condition in which GPR119 and optionally DPP-IV play a role comprising a step of administering to a subject in need thereof an effective amount of a compound of the invention, or a pharmaceutically acceptable salt thereof. Such diseases or conditions diabetes, obesity, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The invention also provides a method for the treatment of type II diabetes, comprising a step of administering to a patient in need thereof an effective amount of a compound of the invention, or a pharmaceutically acceptable salt thereof.

The invention also provides a method for the treatment of obesity, metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of the invention, or a pharmaceutically acceptable salt thereof.

The invention also provides a compound of the invention, or a pharmaceutically acceptable salt thereof, for use in the treatment of a condition as defined above.

The invention also provides the use of a compound of the invention, or a pharmaceutically acceptable salt thereof, in the manufacture of a medicament for the treatment of a condition as defined above.

In the methods of the invention the term “treatment” includes both therapeutic and prophylactic treatment.

The compounds of the invention may exhibit advantageous properties compared to known compounds or combination therapies for the treatment of diabetes.

The compounds of the invention, or pharmaceutically acceptable salts thereof, may be administered alone or in combination with one or more other therapeutically active compounds. The other therapeutically active compounds may be for the treatment of the same disease or condition as the compounds of the invention or a different disease or condition. The therapeutically active compounds may be administered simultaneously, sequentially or separately.

The compounds of the invention may be administered with other active compounds for the treatment of obesity and/or diabetes, for example insulin and insulin analogs, gastric lipase inhibitors, pancreatic lipase inhibitors, sulfonyl ureas and analogs, biguanides e.g. metformin, α2 agonists, glitazones, PPAR-γ agonists, mixed PPAR-α/γ agonists, RXR agonists, fatty acid oxidation inhibitors, α-glucosidase inhibitors, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of the invention, or a pharmaceutically acceptable salt thereof, and at least one other agent, for example another agent for the treatment of diabetes or obesity, represents a further aspect of the invention.

The present invention also provides a method for the treatment of diabetes in a mammal, such as a human, which method comprises administering an effective amount of a compound of the invention, or a pharmaceutically acceptable salt thereof, and another agent, for example another agent for the treatment of diabetes or obesity, to a mammal in need thereof.

The invention also provides the use of a compound of the invention, or a pharmaceutically acceptable salt thereof, and another agent for the treatment of diabetes.

The invention also provides the use of a compound of the invention, or a pharmaceutically acceptable salt thereof, in the manufacture of a medicament for use in combination with another agent, for the treatment of diabetes.

The compound of the invention, or a pharmaceutically acceptable salt thereof, and the other agent(s) may be co-administered or administered sequentially or separately.

Co-administration includes administration of a formulation which includes both the compound of the invention, or a pharmaceutically acceptable salt thereof, and the other agent(s), or the simultaneous or separate administration of different formulations of each agent. Where the pharmacological profiles of the compound of the invention, or a pharmaceutically acceptable salt thereof, and the other agent(s) allow it, coadministration of the two agents may be preferred.

The invention also provides the use of a compound of the invention, or a pharmaceutically acceptable salt thereof, and another agent in the manufacture of a medicament for the treatment of diabetes.

The invention also provides a pharmaceutical composition comprising a compound of the invention, or a pharmaceutically acceptable salt thereof, and another antidiabetic agent, and a pharmaceutically acceptable carrier. The invention also encompasses the use of such compositions in the methods described above.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Materials and Methods

Column chromatography was carried out on SiO₂ (40-63 mesh) unless specified otherwise. LCMS data were obtained as follows: Atlantis 3μ C₁₈ column (3.0×20.0 mm, flow rate=0.85 mL/min) eluting with a H₂O-MeCN solution containing 0.1% HCO₂H over 6 min with UV detection at 220 nm. Gradient information: 0.0-0.3 min 100% H₂O; 0.3-4.25 min: Ramp up to 10% H₂O-90% MeCN; 4.25-4.4 min: Ramp up to 100% MeCN; 4.4-4.9 min: Hold at 100% MeCN; 4.9-6.0 min: Return to 100% H₂O. The mass spectra were obtained using an electrospray ionisation source in either the positive (ES⁺) or negative (ES⁻) ion modes.

LCMS-method 2 data were obtained as follows: Xbridge C18 column (2.1×50 mm, 2.5 μM, flow rate 0.8 mL/min) eluting with an MeCN-10 mM NH₄HCO₃ solution over 1.5 min with UV detection at 215-350 nm. Gradient information: 0-0.8 min: 98% MeCN 2% NH₄HCO₃ to 98% NH₄HCO₃ 2% MeCN; 0.8-1.2 min: hold at 98% NH₄HCO₃ 2% MeCN. The mass spectra were obtained using an electrospray ionisation source in the positive (ES⁺) mode.

LCMS-method 3 data were obtained as follows: Xbridge C18 column (2.1×5.0 mm, 2.55 μM, flow rate 0.8 mL/min) eluting with an MeCN-10 mM NH₄HCO₃ solution over 5 min with UV detection at 215-350 nm. Gradient information: 0-4 min: 98% MeCN 2% NH₄HCO₃ to 98% NH₄HCO₃ 2% MeCN; 4-4.6 min: hold at 98% NH₄HCO₃ 2% MeCN. The mass spectra were obtained using an electrospray ionisation source in the positive (ES⁺) mode.

LCMS-method 4 data were obtained as follows: Xbridge C18 column (3.0×150 mm, 5 μM, flow rate 1.0 mL/min) eluting with an MeCN-10 mM NH₄HCO₃ solution over 5 min with UV detection at 215-350 nm. Gradient information: 0-0.1 min: hold at 5% MeCN 95% NH₄HCO₃; 0.1-3.0 min: 5% MeCN 95% NH₄HCO₃ to 5% NH₄HCO₃ 95% MeCN; 3.0-3.9 min: hold at 5% NH₄HCO₃ 95% MeCN. The mass spectra were obtained using an electrospray ionisation source in the positive (ES⁺) mode.

Chiral-HPLC was performed on a Daicel chiralpak IA 250×20 mm, 5 μM column.

Abbreviations and acronyms: AcOH: Acetic acid; atm: Atmospheres; BA: n-butylamine; CHCl₃: Chloroform; DBU: 1,8-Diazabicyclo[5.4.0]undec-7-ene; DCM: Dichloromethane; DEA: Diethylamine; DIPEA: Diisopropylethylamine; DMAP: Dimethylpyridin-4-ylamine; DMF: Dimethylformamide; DMSO: Dimethylsulfoxide; EDCI: (3-Dimethylaminopropyl)ethylcarbodiimide hydrochloride; EtOAc: Ethyl Acetate; Et₂O: Diethyl ether; EtOH: Ethanol; h: hour(s); HO: Hydrochloric acid; HCO₂H: Formic acid; H₂O: Water; HOBt: 1-Hydroxybenzotriazole monohydrate; HPLC: High performance liquid chromatography; IH: Isohexane; IMS: Industrial methylated spirit; IPA: Isopropyl alcohol; M: Molar; MeCN: Acetonitrile; MeOH: Methanol; MgSO₄: Magnesium sulphate; min: minute/s; MTBE: Methyl-tert-butyl ether; NaHCO₃: Sodium hydrogen carbonate; Na₂CO₃: Sodium carbonate; NaOH: Sodium hydroxide; Na₂SO₄: Sodium sulphate; NH₃: Ammonia; NH₄HCO₃: Ammonium bicarbonate; NH₄OH: Ammonium hydroxide; Pd: Palladium; RT: Retention time; r.t.: Room temperature; sat: saturated; SCX: Strong Cation Exchange resin; SiO₂: Silica gel; THF: Tetrahydrofuran; TFA: Trifluoroacetic acid; TFAA: Trifluoroacetic anhydride; TsOH: p-Toluenesulfonic acid monohydrate

The syntheses of the following compounds have been described elsewhere: 4-(N-hydroxycarbamimidoylmethoxy)piperidine-1-carboxylic acid tert-butyl ester: Bradley et. al., WO2007/003961; 4-((R)-1-Carboxyethoxy)piperidine-1-carboxylic acid tert-butyl ester: Bertram et. al, WO2007/116229; (3S,4S)-3,4-Diazido-1-benzylpyrrolidine: Benbow et. al., WO2007/148185. All other compounds were available from commercial sources.

### Preparation 1

### 4-[5-(6-Chloropyridin-3-yl)-[1,2,4]oxadiazol-3-ylmethoxy]piperidine-1-carboxylic acid tert-butyl ester

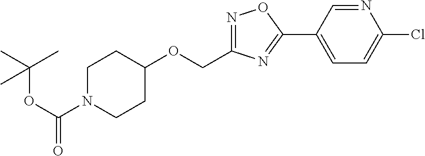

To a solution of 6-chloronicotinic acid (500 mg, 3.17 mmol) in THF (25 mL) was added EDCI (0.74 g, 3.89 mmol), followed by HOBt (583 mg, 3.81 mmol), and the reaction was stirred at r.t. for 10 min. 4-(N-Hydroxycarbamimidoylmethoxy)piperidine-1-carboxylic acid tert-butyl ester (866 mg, 3.17 mmol) was added and the reaction was stirred at r.t. for 16 h before removing the solvent in vacuo. The resulting residue was partitioned between EtOAc (100 mL) and water (50 mL). The organic phase was separated, washed with sat. NaHCO₃ solution, then brine, and dried (MgSO₄), before removal of the solvent in vacuo. The residue was dissolved in toluene and the reaction heated to 110° C. for 16 h. Removal of the solvent in vacuo and purification by column chromatography (IH:EtOAc, 70:30) afforded the title compound: RT=3.97 min, m/z (ES⁺)=395.2 [M+H]⁺.

### Preparation 2

### 4-[5-(2-Chloropyrimidin-5-yl)-[1,2,4]oxadiazol-3-ylmethoxy]piperidine-1-carboxylic acid tert-butyl ester

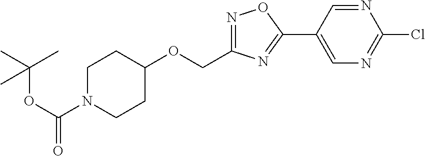

To a solution of 2-chloropyrimidine-5-carboxylic acid (100 mg, 0.63 mmol) in THF (10 mL) was added 1,3-diisopropylcarbodiimide (99 μL, 0.63 mmol) and the reaction was stirred at r.t. for 10 min. 4-(N-Hydroxycarbamimidoylmethoxy)piperidine-1-carboxylic acid tert-butyl ester (172 mg, 0.63 mmol) was added and the mixture was stirred at r.t. for 72 h. The reaction solvent was removed in vacuo and the resulting residue was re-dissolved in EtOAc (100 mL). The organic mixture was washed with water, then brine, and dried (MgSO₄), before removal of the solvent in vacuo. The resulting residue was dissolved in toluene and heated to 80° C. for 16 h. Removal of the solvent in vacuo and purification by column chromatography (IH:EtOAc, 60:40) afforded the title compound: RT=3.77 min, m/z (ES⁺)=396.1 [M+H]⁺.

### Preparation 3

### 4-[3-(6-Chloropyridin-3-yl)-[1,2,4]oxadiazol-5-ylmethoxy]piperidine-1-carboxylic acid tert-butyl ester

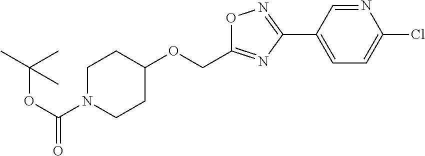

To a solution of 4-carboxymethoxypiperidine-1-carboxylic acid tert-butyl ester (1.51 g, 5.83 mmol) in THF (40 mL) was added EDCI (1.11 g, 6.99 mmol), followed by HOBt (0.95 g, 6.99 mmol), and the reaction was stirred at r.t. for 10 min. 6-Chloro-N-hydroxynicotinamidine (1.00 g, 5.83 mmol) was added and the reaction was stirred at r.t. for 17 h before removing the solvent in vacuo. The resulting residue was partitioned between EtOAc (200 mL) and water (100 mL), then the organic phase was separated, washed with sat. NaHCO₃ solution (100 mL), and dried (MgSO₄) before removal of the solvent in vacuo. The residue was dissolved in toluene and heated to reflux for 20 h before removing the solvent in vacuo and re-dissolving the product in EtOAc (200 mL). The solution was washed with water (100 mL), then brine (50 mL), and dried (MgSO₄). Removal of the solvent in vacuo and purification by recrystallisation from EtOAc/IH afforded the title compound: RT=4.02 min, m/z (ES⁺)=395.1 [M+H]⁺.

### Preparation 4

### 4-{(R)-1-[3-(6-Chloropyridin-3-yl)-[1,2,4]oxadiazol-5-yl]ethoxy}piperidine-1-carboxylic acid tert-butyl ester

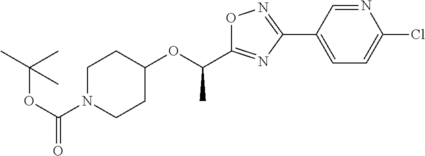

The title compound was prepared by reacting 4-((R)-1-carboxyethoxy)piperidine-1-carboxylic acid tert-butyl ester with 6-chloro-N-hydroxynicotinamidine employing the procedure outlined in Preparation 3: RT=4.20 min, m/z (ES⁺)=409.2 [M+H]⁺.

### Preparation 5

### 4-[5-(6-Chloropyridin-3-yl)-[1,2,4]oxadiazol-3-ylmethoxy]piperidine-1-carboxylic acid isopropyl ester

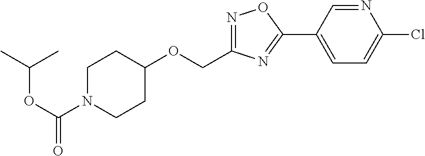

4-[5-(6-Chloropyridin-3-yl)-[1,2,4]oxadiazol-3-ylmethoxy]piperidine-1-carboxylic acid tert-butyl ester (Preparation 1, 578 mg, 1.47 mmol) in a solution of HCl in dioxane (4M, 10 mL) was stirred for 2 h before removal of the solvent in vacuo. To a solution of the product in DCM (20 mL) was added triethylamine (0.66 mL, 4.70 mmol) and the mixture was cooled to 0° C. A solution of isopropyl chloroformate in toluene (1M, 1.76 mL, 1.76 mmol) was added, dropwise, then the resulting reaction was stirred at r.t. for 16 h. The crude mixture was diluted with DCM (100 mL), washed with water (50 mL), sat. Na₂CO₃ solution (50 mL), and brine, then dried (MgSO₄). Removal of the solvent in vacuo afforded the title compound: RT=3.75 min, m/z (ES⁺)=381.2 [M+H]⁺.

The following compounds were prepared from the appropriate tert-butyl carbamate protected compound employing the procedure outlined in Preparation 5:

| Prep | | | |
|---|---|---|---|
| No. | Structure | Name | LCMS |
| 6 | 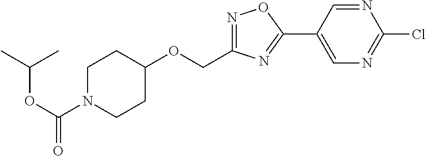 | 4-[5-(6- Chloropyrimidin-5- yl)-[1,2,4]oxadiazol- 3-ylmethoxy]- piperidine-1- carboxylic acid isopropyl ester | RT = 3.54 min, m/z (ES⁺) = 382.1 [M + H]⁺ |
| 7 | 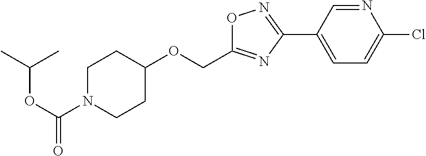 | 4-[3-(6-Chloro- pyridin-3-yl)- [1,2,4]oxadiazol-5- ylmethoxy]piperidine- 1-carboxylic acid isopropyl ester | RT = 3.82 min, m/z (ES⁺) = 381.1 [M + H]⁺ |
| 8 | 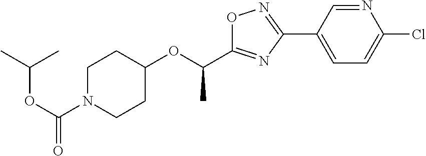 | 4-{(R-1-[3-(6- Chloropyridin-3-yl)- [1,2,4]oxadiazol-5- yl]ethoxy}piperidine- 1-carboxylic acid isopropyl ester | RT = 3.98 min, m/z (ES⁺) = 395.1 [M + H]⁺ |

### Preparation 9

### 4-((R)-1-Carboxyethoxy)piperidine-1-carboxylic acid isopropyl ester

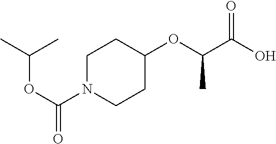

A solution of 4-hydroxypiperidine-1-carboxylic acid isopropyl ester (10.0 g. 53.4 mmol) in THF (100 mL), in an oven dried flask, under argon, was cooled to 0° C. Sodium hydride (60% in mineral oil, 8.55 g, 213.6 mmol) was added, portion-wise, and the resulting mixture was stirred at 0° C. for 1 h before stirring at r.t. for 30 min. To the reaction was added a solution of (S)-2-bromopropionic acid (4.82 g, 53.4 mmol) in THF (40 mL), dropwise, over 30 min, followed by more THF (60 mL), and the mixture was stirred at r.t. for 16 h. The reaction was quenched by the cautious addition of water, then the THF was removed in vacuo. The resulting aqueous mixture was washed with Et₂O and acidified to pH1 with 2M HCl. The mixture was extracted with EtOAc (2×150 mL) then the organic fractions were combined, dried (MgSO₄) and the solvent removed in vacuo to afford the title compound: ¹H NMR δ_{H} (400 MHz, CDCl₃): 4.97-4.86 (m, 1H), 4.19-4.09 (m, 1H), 3.89-3.79 (m, 2H), 3.66-3.58 (m, 1H), 3.18-3.08 (m, 2H), 1.91-1.80 (m, 2H), 1.65-1.50 (m, 2H), 1.47 (d, J=7.0 Hz, 3H), 1.26-1.22 (m, 6H).

### Preparation 10

### 4-((R)-1-Carbamoylethoxy)piperidine-1-carboxylic acid isopropyl ester

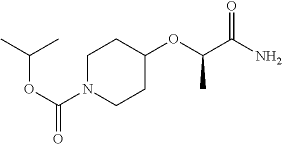

To a solution of 4-((R)-1-carboxyethoxy)piperidine-1-carboxylic acid isopropyl ester (Preparation 9, 200 mg, 0.77 mmol) in THF (10 mL), in an oven-dried flask, under argon, was added EDCI (177 mg, 0.93 mmol), followed by HOBt (126 mg, 0.93 mmol), and the reaction was stirred at r.t. for 10 min. A solution of NH₃ in dioxane (0.5M, 15 μL, 7.72 mmol) was added and the reaction was stirred at r.t. for 24 h. The solvent was removed in vacuo and the resulting residue was partitioned between EtOAc (75 mL) and water (25 mL). The organic layer was removed, washed with sat. NaHCO₃ solution (25 mL), then brine (25 mL), and dried (MgSO₄). Removal of the solvent in vacuo and purification by column chromatography (IH:EtOAc, 5:95, 0:100) afforded the title compound: RT=2.49 min, m/z (ES⁺)=259.2 [M+H]⁺.

### Preparation 11

### 4-((R)-Cyanomethylmethoxy)piperidine-1-carboxylic acid isopropyl ester

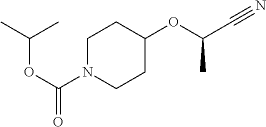

A solution of 4-((R)-1-carbamoylethoxy)piperidine-1-carboxylic acid isopropyl ester (Preparation 10, 200 mg, 0.78 mmol) in THF, in an oven-dried flask, under argon, was cooled to 0° C. To the solution was added triethylamine (320 μL, 2.34 mmol) followed by TFAA (165 μL, 1.16 mmol), and the mixture was stirred at this temperature for 1 h. The reaction was quenched by the addition of water and the mixture extracted with DCM (100 mL). The organic fraction was washed with brine, dried (MgSO₄) and the solvent removed in vacuo to afford the title compound: RT=3.62 min, m/z (ES⁺)=240.1 [M+H]⁺.

### Preparation 12

### 4-[(R)-1-(N-Hydroxycarbamimidoyl)ethoxy]piperidine-1-carboxylic acid isopropyl ester

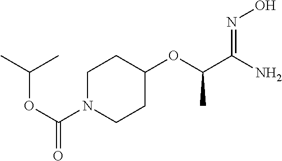

To a solution of 4-((R)-cyanomethylmethoxy)piperidine-1-carboxylic acid isopropyl ester (Preparation 11, 51 mg, 0.02 mmol) in EtOH (5 mL) was added hydroxylamine (50% Wt in water, 470 μL, 0.02 mmol) and the reaction was heated to 80° C. for 16 h. The mixture was concentrated in vacuo and the residue was azeotroped with toluene (×3) to afford the title compound: RT=2.02 min, m/z (ES⁺)=274.1 [M+H]⁺.

The following compounds were prepared by reacting 4-[(R)-1-(N-hydroxycarbamimidoyl)ethoxy]piperidine-1-carboxylic acid isopropyl ester (Preparation 12) with the appropriate carboxylic acid, employing the procedure outlined in Preparation 2:

| Prep | | | |
|---|---|---|---|
| No. | Structure | Name | LCMS |
| 13 | 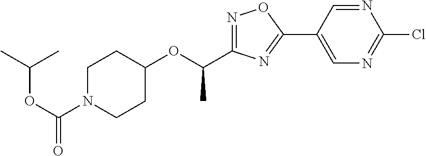 | 4-{(R)-1-[5-(2- Chloropyrimidin-5- yl)-[1,2,4]oxadiazol- 3-yl]ethoxy}- piperidine-1- carboxylic acid isopropyl ester | RT = 3.82 min, m/z (ES⁺) = 396.1 [M + H]⁺ |
| 14 | 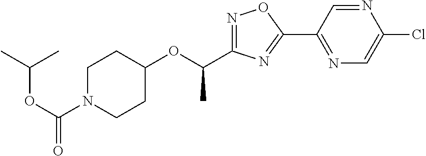 | 4-{(R)-1-[5-(5- Chloropyrazin-2-yl)- [1,2,4]oxadiazol-3- yl]ethoxy}piperidine- 1-carboxylic acid isopropyl ester | RT = 3.88 min, m/z (ES⁺) = 396.1 [M + H]⁺ |

### Preparation 15

### 3-(2,5-Difluorophenyl)-4-nitrobutyric acid methyl ester

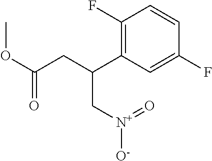

To a solution of (2E)-3-(2,5-difluorophenyl)acrylic acid (21.10 g, 114.7 mmol) in a mixture of DCM and MeOH (DCM:MeOH, 4:1, 250 mL) was added a solution of trimethylsilyldiazomethane (2M in Et₂O, 57.34 mL, 114.7 mmol), over 15 min, and the reaction was stirred at r.t. until complete. AcOH was added, dropwise, until the reaction mixture turned colourless, then the solvent was removed in vacuo. The residue was re-dissolved in MeCN (114 mL), then nitromethane (7.45 mL, 137.6 mmol) was added. The mixture was cooled to 0° C. before adding DBU (17.49 mL, 117.0 mmol), dropwise, over 30 min. The reaction was allowed to warm to r.t. and stirred for 16 h. Removal of the solvent in vacuo and purification by column chromatography (IH:EtOAc, 95:5, 90:10) afforded the title compound: ¹H NMR δ_{H} (400 MHz, CDCl₃): 7.18-7.00 (m, 3H), 4.91-4.77 (m, 2H), 4.27-4.17 (m, 1H), 3.75 (s, 3H), 2.91 (m, 2H).

### Preparation 16

### (trans)-1-Benzyl-4-(2,5-difluorophenyl)-5-nitropiperidin-2-one

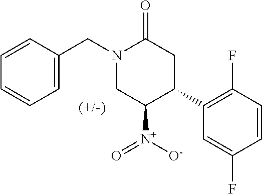

A combination of 3-(2,5-difluorophenyl)-4-nitrobutyric acid methyl ester (Preparation 15, 16.27 g, 62.81 mmol, paraformaldehyde (1.94 g, 64.63 mmol) and benzylamine (13.7 mL, 125.62 mmol) in EtOH was heated to 90° C. in a sealed tube for 16 h. After complete reaction the mixture was partitioned between EtOAc (400 mL) and 2M HCl (600 mL). The organic fraction was separated, washed with brine, dried (MgSO₄), and the solvent removed in vacuo. Purification by column chromatography (IH:EtOAc, 70:30) afforded the title compound: RT=3.72 min m/z (ES⁺)=347.1 [M+H]⁺.

### Preparation 17

### (trans)-1-Benzyl-4-(2,5-difluorophenyl)-3-nitropiperidine hydrochloride HCl

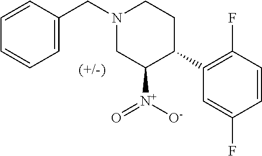

To a solution of (trans)-1-benzyl-4-(2,5-difluorophenyl)-5-nitropiperidin-2-one (Preparation 16, 10.44 g, 30.17 mmol) in THF (90 mL), under argon, was added borane dimethylsulfide complex (2.0M in DCM, 45.3 mL, 90.60 mmol) and the reaction was heated to 70° C. for 3 h. After cooling to r.t. the mixture was diluted with MeOH (20 mL) then 1M HCl (30 mL) was added. The mixture was stirred for 10 min before removal of the solvent in vacuo. Further portions of MeOH (20 mL) and 1M HCl (20 mL) were added and the reaction stirred for 10 min. Removal of the solvent in vacuo afforded the title compound: RT=3.30 min ink (ES⁺)=333.1 [M+H]⁺.

### Preparation 18

### [(3R,4R)-1-Benzyl-4-(2,5-difluorophenyl)piperidin-3-yl]carbamic acid tert-butyl ester

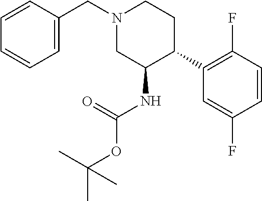

A combination of (trans)-1-benzyl-4-(2,5-difluorophenyl)-3-nitropiperidine hydrochloride (Preparation 17, 11.12 g, 30.17 mmol) and zinc dust (15.69 g, 241.36 mmol) in a mixture of AcOH (55 mL) and EtOH (55 mL) was heated to 80° C. After complete reaction the mixture was filtered and the solvent removed in vacuo. To a solution of the resulting residue in MeOH (30 mL) was added HCl in dioxane (4M, 30 mL), and the solvent was removed in vacuo. The material was triturated with Et₂O (×2), then toluene (×3) to afford the amine as the hydrochloride salt. To a solution of the product in a mixture of THF (150 mL) and water (75 mL), cooled to 0° C., was added triethylamine (12.6 mL, 90.51 mmol), followed by di-tert-butyl dicarbonate (9.59 g, 45.26 mmol). The reaction was allowed to reach r.t. and stirred for 16 h, until complete. The mixture was partitioned between EtOAc (750 mL) and water (200 mL) and the organic phase was separated. The aqueous phase was extracted with EtOAc (500 mL), then the organic fractions were combined, dried (MgSO₄) and the solvent removed in vacuo. Purification by column chromatography (IH:EtOAc, 80:20) and further purification by chiral HPLC (IH:IPA:DEA 90:10:0.1, 15 ml/min, 270 nm, RT=9.8 min) afforded the title compound: RT=2.68 min m/z (ES⁺)=403.2 [M+H]⁺.

### Preparation 19

### [(3R,4R)-4-(2,5-Difluorophenyl)piperidin-3-yl]carbamic acid tert-butyl ester

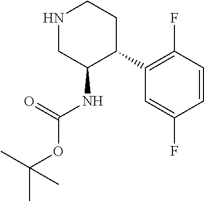

A solution of [(3R,4R)-1-benzyl-4-(2,5-difluorophenyl)piperidin-3-yl]carbamic acid tert-butyl ester (Preparation 18, 1.89 g, 4.70 mmol) in MeOH (94 mL) was passed through an H-Cube apparatus (10% pd/C Catcart 70, 30 bar, 80° C.) at a flow rate of 1 mL per min. The solvent was removed in vacuo to afford the title compound: RT=2.37 min; m/z (ES⁺)=313.2 [M+H]⁺.

### Preparation 20

### 2-Methoxypyrimidine-5-carbonitrile

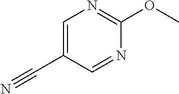

To a solution of 5-bromo-2-methoxypyrimidine (2.55 g, 13.49 mmol) and zinc cyanide (1.90 g, 16.19 mmol) in DMF (40 mL), in an oven-dried flask, was added palladium tetrakis (4.68 g, 4.05 mmol) and the mixture was bubbled with argon for 10 min before being heated to 85° C. until complete. The reaction solvent was concentrated in vacuo and the residue was partitioned between EtOAc (300 mL) and water (200 mL). The organic layer was separated, then the aqueous layer was extracted with EtOAc (2×150 mL). Organic fractions were combined, washed with sat. NaHCO₃ solution (2×100 mL), brine (100 mL), and dried (MgSO₄). Removal of the solvent in vacuo and purification by column chromatography (IH:DCM, 1:9, DCM, DCM:MeOH, 95:5) afforded the title compound: ¹H NMR δ_{H} (400 MHz, CDCl₃): 8.80 (s, 2H), 4.12 (s, 3H)

### Preparation 21

### N-Hydroxy-2-methoxypyrimidine-5-carboxamidine

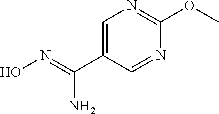

To a solution of 2-methoxypyrimidine-5-carbonitrile (Preparation 20, 1.21 g, 9.01 mmol) in EtOH (50 mL) was added hydroxylamine (50% Wt in water, 0.65 mL, 9.91 mmol) and the reaction was heated to 65° C. for 16 h. The mixture was concentrated in vacuo to afford the title compound: RT=0.85 min, m/z (ES⁺)=169.0 [M+H]⁺.

### Preparation 22

### 4-[3-(2-Methoxypyrimidin-5-yl)-[1,2,4]oxadiazol-5-ylmethoxy]piperidine-1-carboxylic acid tert-butyl ester

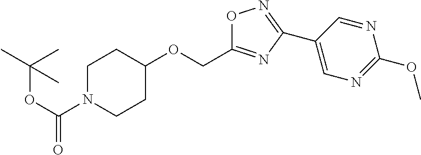

To a solution of 4-carboxymethoxypiperidine-1-carboxylic acid tert-butyl ester (1.08 g, 4.16 mmol) in THF (30 mL) was added EDCI (0.96 g, 5.00 mmol) followed by HOBt (0.77 g, 5.00 mmol), and the reaction was stirred at r.t. for 15 min. N-Hydroxy-2-methoxypyrimidine-5-carboxamidine (Preparation 21, 0.70 g, 4.16 mmol) was added and the reaction was stirred at r.t. for 16 h before removing the solvent in vacuo. The resulting residue was partitioned between EtOAc (50 mL) and water (50 mL), then the organic phase was separated, washed with sat. NaHCO₃ solution (50 mL), brine (50 mL), and dried (MgSO₄), before removal of the solvent in vacuo. The residue was dissolved in toluene (30 mL) and heated to 85° C. until completion. Removal of the solvent in vacuo afforded the title compound: RT=3.58 min, m/z (ES⁺)=392.2 [M+H]⁺.

### Preparation 23

### 4-[3-(2-Methoxypyrimidin-5-yl)-[1,2,4]oxadiazol-5-ylmethoxy]piperidine-1-carboxylic acid isopropyl ester

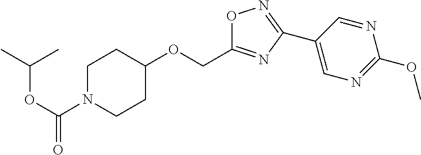

To a solution of 4-[3-(2-methoxypyrimidin-5-yl)-[1,2,4]oxadiazol-5-ylmethoxy]piperidine-1-carboxylic acid tert-butyl ester (Preparation 22, 1.33 g, 3.40 mmol) in dioxane (10 mL) was added HCl in dioxane (4M, 2.55 mL, 10.19 mmol) and the reaction was stirred at r.t. for 4 h. A further portion of HCl in dioxane (7.5 mL, 30 mmol) was added and stirring continued for 72 h. Removal of the solvent in vacuo afforded the intermediate product 2-methoxy-5-[5-(piperidin-4-yloxymethyl)-[1,2,4]oxadiazol-3-yl]pyrimidine hydrochloride: RT=1.95 min, m/z (ES⁺)=292.1 [M+H]⁺.

To a suspension of the product in DCM (20 mL) was added triethylamine (0.99 mL, 7.11 mmol) and the mixture was cooled to 0° C. Isopropyl chloroformate (1M in toluene, 3.73 mL, 3.73 mmol) was added, dropwise, over 20 min, and the reaction was allowed to stir for a further 2 h. The mixture was partitioned between DCM (50 mL) and water (50 mL) and the organic phase was separated. The aqueous phase was extracted with DCM (100 mL) then organic fractions were combined, washed with brine (100 mL), dried (MgSO₄), and the solvent removed in vacuo. Purification by column chromatography (DCM:MeOH, 98:2, 97:3, 96:4) afforded the title compound: RT=3.44 min, m/z (ES⁺)=378.2 [M+H]⁺.

### Preparation 24

### 4-[3-(2-Hydroxypyrimidin-5-yl)-[1,2,4]oxadiazol-5-ylmethoxy]piperidine-1-carboxylic acid isopropyl ester

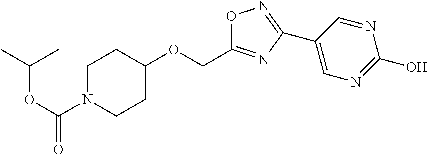

To a solution of 4-[3-(2-methoxypyrimidin-5-yl)-[1,2,4]oxadiazol-5-ylmethoxy]piperidine-1-carboxylic acid isopropyl ester (Preparation 23, 885 mg, 2.34 mmol) in MeCN (30 mL), under an atmosphere of argon, was added sodium iodide (1054 mg, 7.03 mmol) followed by trimethylsilyl chloride (893 μL, 7.03 mmol), and the reaction was heated to 65° C. for 16 h. The mixture was partitioned between EtOAc (100 mL) and sat. sodium thiosulfate solution (100 mL), and the organic phase was separated. The aqueous phase was extracted with EtOAc (2×50 mL) then organic fractions were combined, washed with brine (100 mL), dried (MgSO₄), and the solvent removed in vacuo. Purification by column chromatography (DCM:MeOH, 100:0, 98:2, 96:4, 94:6) afforded the title compound: RT=2.76 min, m/z (ES⁺)=364.2 [M+H]⁺.

### Preparation 25

### 4-[3-(2-Chloropyrimidin-5-yl)-[1,2,4]oxadiazol-5-ylmethoxy]piperidine-1-carboxylic acid isopropyl ester

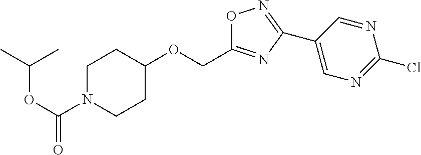

To a solution of 4-[3-(2-hydroxypyrimidin-5-yl)-[1,2,4]oxadiazol-5-ylmethoxy]piperidine-1-carboxylic acid isopropyl ester (Preparation 24, 199 mg, 0.55 mmol) in phosphorus oxychloride (6 mL), under argon, was added N,N-dimethylaminoaniline (90 μL, 0.71 mmol) and the mixture was heated to 50° C. for 5 h. The reaction was quenched by being added, dropwise, to ice (50 mL) and the organics were extracted into DCM (3×50 mL). The organic fractions were combined, washed with brine (50 mL), dried (MgSO₄), and the solvent removed in vacuo. Purification by column chromatography (DCM:MeOH, 97:3) afforded the title compound: RT=3.71 min, m/z (ES⁺)=382.1 [M+H]⁺.

### Preparation 26

### 4-{(R)-1-[3-(2-Methoxypyrimidin-5-yl)-[1,2,4]oxadiazol-5-yl]ethoxy}piperidine-1-carboxylic acid isopropyl ester

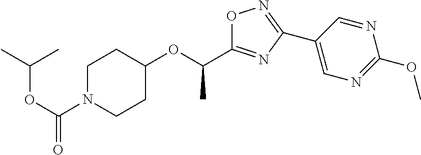

4-((R)-1-Carboxyethoxy)piperidine-1-carboxylic acid isopropyl ester (Preparation 9) was reacted with N-hydroxy-2-methoxypyrimidine-5-carboxamidine (Preparation 21) employing the procedure outlined in Preparation 22. Purification by column chromatography (DCM:MeOH, 97:3) afforded the title compound: RT=3.55 min, m/z (ES⁺)=392.2 [M+H]⁺.

### Preparation 27

### 4-{(R)-1-[3-(2-Hydroxypyrimidin-5-yl)-[1,2,4]oxadiazol-5-yl]ethoxy}piperidine-1-carboxylic acid isopropyl ester

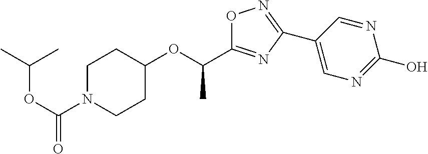

The title compounds was prepared from 4-{(R)-1-[3-(2-methoxypyrimidin-5-yl)-[1,2,4]oxadiazol-5-yl]ethoxy}piperidine-1-carboxylic acid isopropyl ester (Preparation 26) employing the procedure outlined in Preparation 24: RT=2.87 min, m/z (ES⁺)=378.2 [M+H]⁺.

### Preparation 28

### 4-{(R)-1-[3-(2-Chloropyrimidin-5-yl)-[1,2,4]oxadiazol-5-yl]ethoxy}-piperidine-1-carboxylic acid isopropyl ester

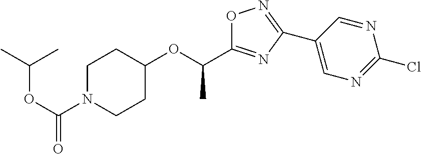

The title compound was prepared from 4-{(R)-1-[3-(2-hydroxypyrimidin-5-yl)-[1,2,4]oxadiazol-5-yl]ethoxy}piperidine-1-carboxylic acid isopropyl ester (Preparation 27) employing the procedure outlined in Preparation 25: RT=3.82 min, m/z (ES⁺)=396.1 [M+H]⁺.

### Preparation 29

### (trans)-3-(9H-Fluoren-9-ylmethoxycarbonylamino)-4-(2-fluorophenyl)pyrrolidine-1-carboxylic acid tert-butyl ester

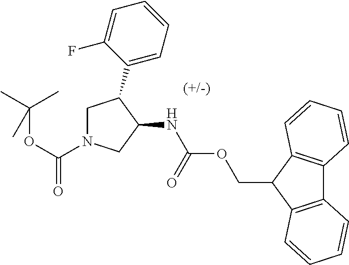

To a solution of (trans)-3-amino-4-(2-fluorophenyl)pyrrolidine-1-carboxylic acid tert-butyl ester (2.00 g, 7.13 mmol) and triethylamine (1.59 mL, 11.40 mmol) in a combination of dioxane and water (2:1, 75 mL), cooled to 0° C., was added 9-fluorenylmethyl chloroformate (2.31 g, 8.92 mmol). The reaction was allowed to reach r.t. before stirring for 16 h. The mixture was diluted with EtOAc, then the solution was washed with water, 1M HCl, sat. NaHCO₃ solution, brine, and dried (MgSO₄). Removal of the solvent in vacuo and purification by column chromatography (IH:EtOAc, 90:10, 80:20, 70:30) afforded the title compound: RT=4.28 min m/z (ES⁺)=503.3 [M+H]⁺.

### Preparation 30

### [(trans)-4-(2-Fluorophenyl)pyrrolidin-3-yl]carbamic acid 9H-fluoren-9-ylmethyl ester hydrochloride

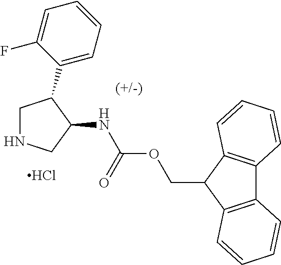

To a solution of (trans)-3-(9H-fluoren-9-ylmethoxycarbonylamino)-4-(2-fluorophenyl)pyrrolidine-1-carboxylic acid tert-butyl ester (Preparation 29, 1.50 g, 2.98 mmol) in dioxane (30 mL) was added a solution of HCl in dioxane (4M, 30 mL) and the reaction was stirred at r.t. for 16 h, after which time a precipitate had formed. Et₂O was added to the mixture until no further precipitation was observed, and the solvent was decanted. The residue was suspended in a further volume of Et₂O and the mixture was stirred for 5 min before decanting the solvent. This process was repeated twice more and the resulting residue was concentrated to dryness to afford the title compound: RT=2.82 min m/z (ES⁺)=403.1 [M+H]⁺.

### Preparation 31

### (3R,4S)-3-(9H-Fluoren-9-ylmethoxycarbonylamino)-4-(2-fluorophenyl)pyrrolidine-1-carboxylic acid tert-butyl ester

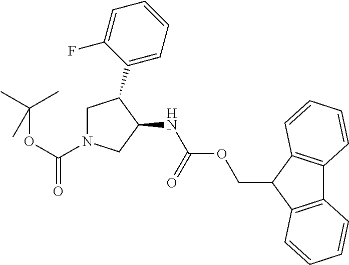

The title compound was afforded via chiral HPLC separation of (trans)-3-(9H-fluoren-9-ylmethoxycarbonylamino)-4-(2-fluorophenyl)pyrrolidine-1-carboxylic acid tert-butyl ester (Preparation 29): IH:CHCl₃:IPA:DEA 85:10:5:0.1, 15 mL/min, 270 nm, RT=9.4 min.

### Preparation 32

### [(3R,4S)-4-(2-Fluorophenyl)pyrrolidin-3-yl]carbamic acid 9H-fluoren-9-ylmethyl ester hydrochloride

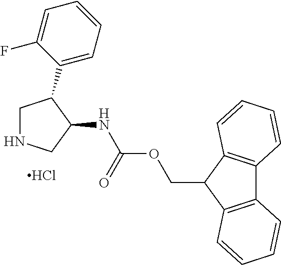

The title compound was prepared from (3R,4S)-3-(9H-fluoren-9-ylmethoxycarbonylamino)-4-(2-fluorophenyl)pyrrolidine-1-carboxylic acid tert-butyl ester (Preparation 31) employing the procedure outlined in Preparation 30: RT=2.82 min m/z (ES⁺)=403.1 [M+H]⁺.

### Preparation 33

### 2,4-Difluoro-1-((E)-2-nitrovinyl)benzene

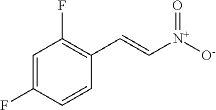

To a solution of 2,4-difluorobenzaldehyde (25.0 g, 0.18 mol) and nitromethane (11.4 mL, 0.21 mol) in MeOH (53 mL), under argon, cooled to −15° C., was added a solution of NaOH (7.4 g, 0.19 mol) in water (26 mL), dropwise, over 20 min. The resulting mixture was stirred at −15° C. and a precipitate formed after 30 min. More MeOH was added to form a slurry and stirring continued for 15 min before allowing the reaction to warm to 0° C. Ice water was added and the mixture was stirred for 15 min before adding 4M HCl (100 mL). The organic fraction was extracted with DCM (3×300 mL), dried (Na₂SO₄) and the solvent removed in vacuo. A portion of the residue (10.0 g, 50 mmol) was dissolved in acetic anhydride (8.1 mL, 90 mol) and cooled to 0° C. under argon. DMAP (0.4 g, 3 mmol) was added and the reaction was stirred at this temperature for 20 min before warming the mixture to r.t. and allowing it to stir for a further 16 h. The reaction solvent was removed in vacuo and the resulting residue was re-dissolved in DCM. Remaining acetic anhydride was destroyed by the addition of a small volume of 1M NaOH solution, then the resulting solution was dried (MgSO₄) and concentrated in vacuo. Purification by column chromatography (DCM) afforded the title compound: RT=3.60 min; m/z (ES⁺)=186.1 [M+H]⁺.

### Preparation 34

### (trans)-1-Benzyl-3-(2,4-difluorophenyl)-4-nitropyrrolidine

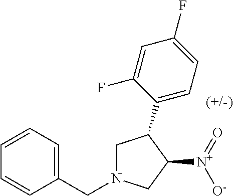

A solution of 2,4-difluoro-1-((E)-2-nitrovinyl)benzene (Preparation 33, 8.0 g, 43.0 mmol) in DCM (250 mL), under argon, was cooled to −30° C. N-(Methoxymethyl)-N-(trimethylsilylmethyl)benzylamine (11.7 mL, 45.0 mmol) was added so as to maintain the temperature at −30° C. The reaction was stirred for 10 min before the dropwise addition of TFA (0.3 mL, 4.3 mmol), and the resulting mixture was allowed to stir at r.t. over 16 h. The reaction mixture was washed with water, then brine, and dried (Na₂SO₄). Removal of the solvent in vacuo to afford the title compound: RT=3.05 min; m/z (ES⁺)=319.1 [M+H]⁺.

### Preparation 35

### [(trans)-1-Benzyl-4-(2,4-difluorophenyl)pyrrolidin-3-yl]carbamic acid tert-butyl ester

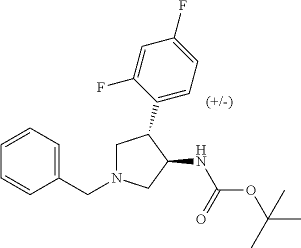

A combination of (trans)-1-benzyl-3-(2,4-difluorophenyl)-4-nitropyrrolidine (Preparation 34, 25.0 g, 0.08 mol) and zinc dust (17.8 g, 0.28 mol) in a mixture of AcOH and EtOH (1:1, 500 mL) was heated to 70° C. After 45 h a further portion of zinc dust (12.0 g, 0.18 mol) was added and heating continued for 20 min. After complete reaction the solvent was removed in vacuo. The resulting residue was re-dissolved in EtOAc, washed with sat. NaHCO₃ solution, then brine, and dried (Na₂SO₄). Removal of the solvent in vacuo afforded the intermediate product (trans)-1-benzyl-4-(2,4-difluorophenyl)pyrrolidin-3-ylamine: RT=1.82 min; m/z (ES⁺)=289.1 [M+H]⁺. To a solution of the product in THF (400 mL), under argon, was added triethylamine (20.4 mL, 0.15 mol) and the solution was cooled to 0° C. Di-tert-butyl dicarbonate (19.0 g, 0.09 mol) was added over 5 min, and the reaction was allowed to reach r.t. over 16 h. The solvent was removed in vacuo, then the resulting residue was re-dissolved in EtOAc, washed with brine, dried (Na₂SO₄), and the solvent removed in vacuo. To the product was added heptane (100 mL), and the suspension was sonicated until fully dissolved. The solution was allowed to stand for 60 h, allowing formation of a precipitate. The solvent was decanted and the solids were washed with a fresh portion of heptane (50 mL) to afford the title compound: RT=2.74 min; m/z (ES⁺)=389.3 [M+H]⁺.

### Preparation 36

### [(3R,4S)-1-Benzyl-4-(2,4-difluorophenyl)pyrrolidin-3-yl]carbamic acid tert-butyl ester

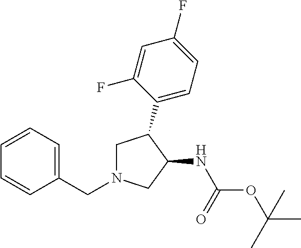

The title compound was afforded via chiral HPLC separation of [(trans)-1-benzyl-4-(2,4-difluorophenyl)pyrrolidin-3-yl]carbamic acid tert-butyl ester (Preparation 35): IH:IPA:DEA 96:4:0.1, 15 mL/min, 270 nm, RT=9.8 min.

### Preparation 37

### [(3R,4S)-4-(2,4-Difluorophenyl)pyrrolidin-3-yl]carbamic acid tert-butyl ester

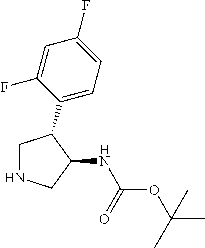

The title compound was prepared from [(3R,4S)-1-benzyl-4-(2,4-difluorophenyl)pyrrolidin-3-yl]carbamic acid tert-butyl ester (Preparation 36) employing the procedure outlined in Preparation 19, but at a pressure of 10 bar and temperature of 50° C.: RT=2.38 min; m/z (ES⁺)=299.1 [M+H]⁺.

### Preparation 38

### 4-((R)-1-{5-[6-(Benzotriazol-1-yloxy)pyridazin-3-yl]-[1,2,4]oxadiazol-3-yl}ethoxy)piperidine-1-carboxylic acid isopropyl ester

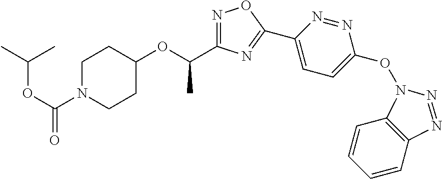

To a solution of 6-chloropyridazine-3-carboxylic acid (60 mg, 0.38 mmol) in THF (10 mL) was added EDCI (107 mg, 0.46 mmol), followed HOBt (69 mg, 0.46 mmol), and the reaction was stirred at r.t. for 15 min 4-[(R)-1-(N-Hydroxycarbamimidoyl)ethoxy]piperidine-1-carboxylic acid isopropyl ester (Preparation 12, 103 mg, 0.38 mmol) was added and stirring continued for 4 h, before removing the solvent in vacuo. The resulting residue was partitioned between EtOAc and water, then the organic phase was separated, washed with sat. NaHCO₃ solution, dried (MgSO₄) and the solvent removed in vacuo. The residue was dissolved in toluene and heated to reflux for 16 h before concentrating the solvent in vacuo and redissolving the product in EtOAc. The solution was washed with water, then brine, and dried (MgSO₄), before removal of the solvent in vacuo. Purification by column chromatography (IH:EtOAc, 1:1) afforded the title compound: RT=3.87 min, m/z (ES⁺)=495.2 [M+H]⁺.

### Preparation 39

### 3-((R)-1-Carboxyethoxy)azetidine-1-carboxylic acid tert-butyl ester

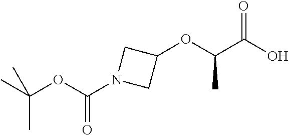

A solution of 3-hydroxyazetidine-1-carboxylic acid tert-butyl ester (3.00 g, 17.32 mmol) in THF (50 mL), under argon, was cooled to 0° C. and sodium hydride (60% in mineral oil, 0.76 g, 18.98 mmol) was added, portionwise, over 10 min. The reaction was stirred at r.t. for 72 h, then water (50 mL) was added. The mixture was washed with EtOAc (2×100 mL) and the resulting aqueous solution was acidified to pH1 with 2M HCl. The mixture was extracted with EtOAc (3×80 mL) then organic fractions were combined, washed with brine (100 mL), dried (MgSO₄) and the solvent removed in vacuo to afford the title compound: ¹H NMR δ_{H} (400 MHz, CDCl₃): 4.40-4.31 (m, 1H), 4.16-4.08 (m, 2H), 4.04-3.93 (m, 2H), 3.90-3.85 (m, 1H), 1.48 (d, T=7.0 Hz, 3H), 1.44 (s, 9H).

### Preparation 40

### 3-((R)-1-Methoxycarbonylethoxy)azetidine-1-carboxylic acid tert-butyl ester

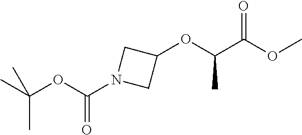

To a solution of 3-((R)-1-carboxyethoxy)azetidine-1-carboxylic acid tert-butyl ester (Preparation 39, 3.29 g, 13.40 mmol) in a mixture of toluene and MeOH (4:1, 25 mL), cooled to 0° C., was added trimethylsilyldiazomethane (2M in hexanes, 8.71 mL, 17.42 mmol), dropwise, over 5 min. The reaction was stirred at this temperature for 30 min before being quenched by the addition of AcOH (1 mL). Removal of the solvent in vacuo and purification by column chromatography (DCM:MeOH, 100:0, 98:2, 95:5) afforded the title compound: ¹H NMR δ_{H} (400 MHz, CDCl₃): 4.35-4.27 (m, 1H), 4.13-4.04 (m, 2H), 3.99-3.91 (m, 2H), 3.87-3.82 (m, 1H), 3.75 (s, 3H), 1.44-1.41 (m, 12H).

### Preparation 41

### (R)-2-(Azetidin-3-yloxy)propionic acid methyl ester trifluoroacetic acid salt

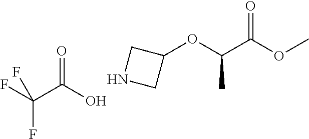

To a solution of 3-((R)-1-methoxycarbonylethoxy)azetidine-1-carboxylic acid tert-butyl ester (Preparation 40, 2.69 g, 10.37 mmol) in DCM (50 mL), under argon, cooled to 0° C., was added TFA (10 mL) and the reaction was stirred at this temperature for 1 h. A further portion of TFA (2 mL) was added and stirring continued for 30 min. Removal of the solvent in vacuo afforded the title compound: ¹H NMR δ_{H} (400 MHz, DMSO-d₆): 4.51-4.43 (m, 1H), 4.24-4.17 (m, 1H), 4.16-4.08 (m, 2H), 3.96-3.83 (m, 2H), 3.66 (s, 3H), 1.30 (d, J=6.6 Hz, 3H).

### Preparation 42

### 3-((R)-1-Methoxycarbonylethoxy)azetidine-1-carboxylic acid isopropyl ester

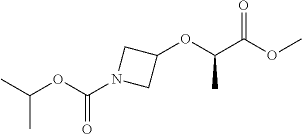

A suspension of (R)-2-(azetidin-3-yloxy)propionic acid methyl ester trifluoroacetic acid salt (Preparation 41, 1.42 g, 5.19 mmol) in DCM (20 mL), under argon, was cooled to 0° C. Triethylamine (1.66 mL, 11.93 mmol) was added, followed by a solution of isopropyl chloroformate (1M in toluene, 6.22 mL, 6.22 mmol), dropwise, and the reaction was stirred at this temperature for 2 h. The mixture was diluted with DCM (30 mL), washed with 1M HCl (30 mL), sat. NaHCO₃ solution (30 mL), then brine (30 mL), and dried (MgSO₄). Removal of the solvent in vacuo and purification by column chromatography (DCM:MeOH, 99:0, 98:2) afforded the title compound: ¹H NMR δ_{H} (400 MHz, CDCl₃): 4.94-4.86 (m, 1H), 4.38-4.30 (m, 1H), 4.17-4.09 (m, 2H), 4.01-3.94 (m, 2H), 3.91-3.86 (m, 1H), 3.76 (s, 3H), 1.43 (d, J=7.0 Hz, 3H), 1.22 (d, J=6.2 Hz, 6H).

### Preparation 43

### 3-((R)-1-Carboxyethoxy)azetidine-1-carboxylic acid isopropyl ester

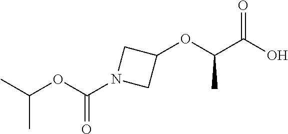

To a solution of 3-((R)-1-methoxycarbonylethoxy)azetidine-1-carboxylic acid isopropyl ester (Preparation 42, 829 mg, 3.38 mmol) in THF (6 mL), cooled to 0° C., was added water (1.5 mL) followed by lithium hydroxide monohydrate (298 mg, 7.10 mmol), and the resulting mixture was stirred at this temperature for 3 h. The reaction solvent was concentrated in vacuo, then water (15 mL) was added and the solution washed with EtOAc (20 mL). The aqueous phase was acidified to pH 1 with 1M HCl and extracted with EtOAc (2×30 mL). The organic fractions were combined, washed with water (20 mL), dried (MgSO₄), and the solvent removed in vacuo to afford the title compound: ¹H NMR δ_{H} (400 MHz, DMSO-d₆): 4.77-4.66 (m, 1H), 4.39-4.31 (m, 1H), 4.08-4.00 (m, 2H), 3.99-3.92 (m, 1H), 3.77-3.66 (m, 2H), 1.28 (d, J=6.6 Hz, 3H), 1.15 (d, 6H).

### Preparation 44

### 2,5-Difluoro-1-((E)-2-nitrovinyl)benzene

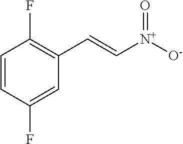

The title compound was prepared from 2,5-difluorobenzaldehyde employing a similar method to that outlined in Preparation 33. After reaction with DMAP the crude mixture was diluted with sat. NaHCO₃ solution. The precipitate that formed was stirred for 30 min, filtered, and dried to afford the title compound: ¹H NMR δ_{H} (300 MHz, CDCl₃): 8.00-7.96 (m, 1H), 7.71-7.66 (m, 1H), 7.25-7.11 (m, 3H).

### Preparation 45

### (trans)-1-Benzyl-3-(2,5-difluorophenyl)-4-nitropyrrolidine

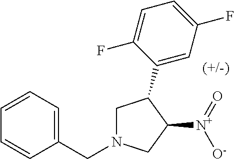

The title compound was prepared from 2,5-difluoro-1-((E)-2-nitrovinyl)benzene (Preparation 44) employing the method outlined in Preparation 34, but the reaction was carried out at 0° C. Purification by column chromatography (Hexane:EtOAc, 100:0, 98:2, 95:5, 90:10) afforded the title compound. LCMS method 2: RT=0.96 min; m/z (ES⁺)=319.2 [M+H]⁺.

### Preparation 46

### [(trans)-1-Benzyl-4-(2,5-difluorophenyl)pyrrolidin-3-yl]carbamic acid tert-butyl ester

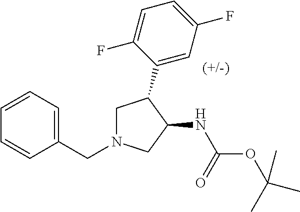

A combination of (trans)-1-benzyl-3-(2,5-difluorophenyl)-4-nitropyrrolidine (Preparation 45, 45.6 g, 0.14 mol) and zinc dust (57.0 g, 0.86 mol) in a mixture of AcOH and EtOH (1:1, 820 mL) was heated to 65° C. After complete reaction the mixture was filtered, washing with AcOH, and the filtrate was concentrated in vacuo. The resulting residue was re-dissolved in EtOAc, washed with sat. NaHCO₃ solution, then brine, and dried (Na₂SO₄). Removal of the solvent in vacuo and purification by column chromatography (DCM:MeOH:NH₃, 100:0:0, 95:5:0, 90:10:0, 90:10:5) afforded the intermediate product (trans)-1-benzyl-4-(2,4,5-trifluorophenyl)pyrrolidin-3-ylamine LCMS method 2: RT=0.80 min; m/z (ES⁺)=289.4 [M+H]⁺. To a solution of the product (12.2 g, 42.3 mmol) in THF (250 mL), under argon, was added triethylamine (12.0 mL, 84.6 mol) and the mixture was cooled to 0° C. Di-tert-butyl dicarbonate (11.0 g, 50.4 mol) was added over 5 min, then the reaction was allowed to reach r.t. and stirred for 16 h. The solvent was concentrated, then the resulting residue was re-dissolved in EtOAc, washed with brine, dried (Na₂SO₄), and the solvent removed in vacuo. The product was triturated several times with heptane to afford the title compound. LCMS method 3: RT=3.08 min; m/z (ES⁺)=389.5 [M+H]⁺.

### Preparation 47

### [(3R,4S)-1-Benzyl-4-(2,5-difluorophenyl)pyrrolidin-3-yl]carbamic acid tert-butyl ester

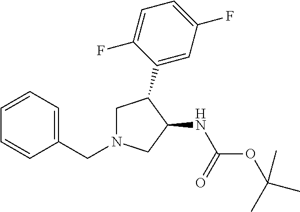

The title compound was afforded via chiral HPLC separation of [(trans)-1-benzyl-4-(2,5-difluorophenyl)pyrrolidin-3-yl]carbamic acid tert-butyl ester (Preparation 46): IH:IPA:DEA 96:4:0.1, 15 mL/min, 270 nm, RT=10.9 min.

### Preparation 48

### [(3R,4S)-4-(2,5-Difluorophenyl)pyrrolidin-3-yl]carbamic acid tert-butyl ester

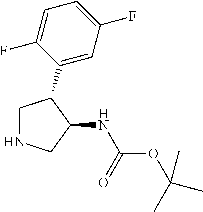

The title compound was prepared from [(3R,4S)-1-benzyl-4-(2,5-difluorophenyl)pyrrolidin-3-yl]carbamic acid tert-butyl ester (Preparation 47) employing the procedure outlined in Preparation 19, but the reaction was carried out at 90° C. RT=2.35 min; m/z (ES⁺)=299.2 [M+H]⁺.

### Preparation 49

### [(3R,4S)-1-(5-Cyanopyrimidin-2-yl)-4-(2,5-difluorophenyl)pyrrolidin-3-yl]carbamic acid tert-butyl ester

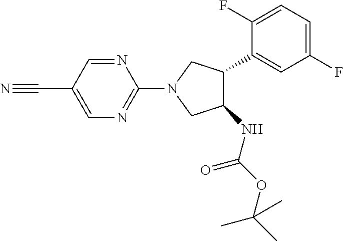

To a mixture of 2-chloropyrimidine-5-carbonitrile (71 mg, 0.51 mmol) and [(3R,4S)-4-(2,5-difluorophenyl)pyrrolidin-3-yl]carbamic acid tert-butyl ester (Preparation 48, 160 mg, 0.54 mmol) in DMSO (1 mL) was added DBU (76 μL, 0.51 mmol). The mixture was bubbled with argon for 5 min and then heated to 70° C. for 16 h. After cooling to r.t. the crude reaction mixture was partitioned between EtOAc (50 mL) and water (50 mL), and the organic phase was separated. The aqueous phase was extracted further with EtOAc (20 mL) then the organic fractions were combined, washed with water (30 mL), sat. NaHCO₃ solution (50 mL), then brine (50 mL), and dried (MgSO₄). Removal of the solvent in vacuo and purification by column chromatography (DCM:MeOH, 100:0, 98:2) afforded the title compound: RT=4.03 min; m/z (ES⁺)=402.1 [M+H]⁺.

### Preparation 50

### {(3R,4S)-4-(2,5-Difluorophenyl)-1-[5-(N-hydroxycarbamimidoyl)-pyrimidin-2-yl]pyrrolidin-3-yl}carbamic acid tert-butyl ester

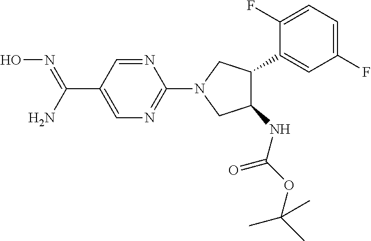

The title compound was prepared from [(3R,4S)-1-(5-cyanopyrimidin-2-yl)-4-(2,5-difluorophenyl)pyrrolidin-3-yl]carbamic acid tert-butyl ester (Preparation 49) employing the procedure outlined in Preparation 21: RT=2.72 min; m/z (ES⁺)=435.2 [M+H]⁺.

### Preparation 51

### 3-[(R)-1-(3-{2-[(3R,4S)-3-tert-Butoxycarbonylamino-4-(2,5-difluorophenyl)pyrrolidin-1-yl]pyrimidin-5-yl}-[1,2,4]oxadiazol-5-yl)ethoxy]azetidine-1-carboxylic acid isopropyl ester

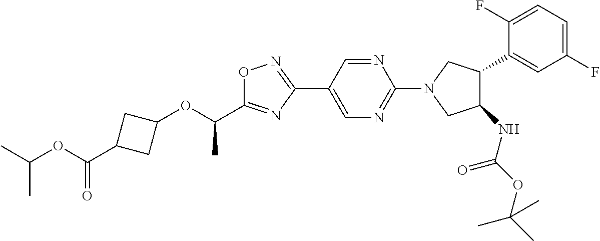

3-((R)-1-Carboxyethoxy)azetidine-1-carboxylic acid isopropyl ester (Preparation 43) was reacted with {(3R,4S)-4-(2,5-difluorophenyl)-1-[5-(N-hydroxycarbamimidoyl)pyrimidin-2-yl]pyrrolidin-3-yl}carbamic acid tert-butyl ester (Preparation 50) employing the procedure outlined in Preparation 22. Purification by column chromatography (IH:EtOAc, 6:4) afforded the title compound: RT=4.44 min, m/z (ES⁺)=630.2 [M+H]⁺.

### Preparation 52

### [(3R,4S)-1-(5-Cyanopyrazin-2-yl)-4-(2,5-difluorophenyl)pyrrolidin-3-yl]carbamic acid tert-butyl ester

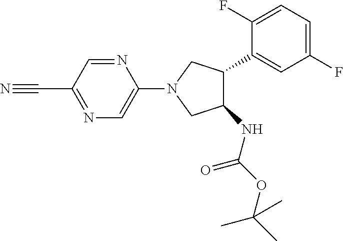

To a mixture of 5-chloropyrazine-2-carbonitrile (140 mg, 1.01 mmol) and [(3R,4S)-4-(2,5-difluorophenyl)pyrrolidin-3-yl]carbamic acid tert-butyl ester (Preparation 48, 300 mg, 1.01 mmol) in DMSO (1 mL) was added DBU (150 μL, 1.01 mmol). The mixture was bubbled with argon for 5 min and then heated to 70° C. for 3 h. After cooling to r.t. the crude reaction mixture was partitioned between EtOAc (200 mL) and water (75 mL), then the organic phase was separated, washed with brine, dried (MgSO₄), and the solvent removed in vacuo. Purification by column chromatography (IH:EtOAc, 60:40) afforded the title compound: RT=3.93 min; m/z (ES⁺)=402.1 [M+H]⁺.

### Preparation 53

### {(3R,4S)-4-(2,5-Difluorophenyl)-1-[5-(N-hydroxycarbamimidoyl)pyrazin-2-yl]pyrrolidin-3-yl}carbamic acid tert-butyl ester

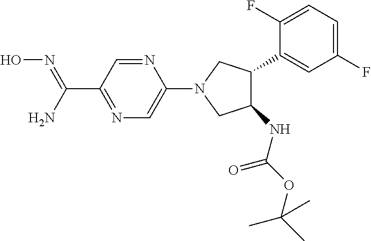

The title compound was prepared from [(3R,4S)-1-(5-cyanopyrazin-2-yl)-4-(2,5-difluorophenyl)pyrrolidin-3-yl]carbamic acid tert-butyl ester (Preparation 52) employing the procedure outlined in Preparation 12, but the reaction was only heated for 3 h: RT=2.73 min; m/z (ES⁺)=435.2 [M+H]⁺.

### Preparation 54

### [(trans)-1-Benzyl-4-(2,4,5-trifluorophenyl)pyrrolidin-3-yl]carbamic acid tert-butyl ester

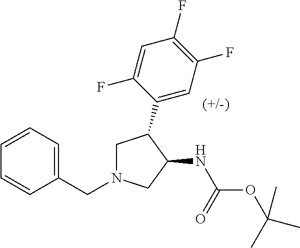

The title compound was prepared in 3 steps from 2,4,5-trifluorobenzaldehyde employing the procedures outlined in the synthesis of Preparation 46. LCMS method 3: RT=3.10 min; m/z (ES⁺)=407.3 [M+H]⁺.

### Preparation 55

### [(trans)-4-(2,4,5-Trifluorophenyl)pyrrolidin-3-yl]carbamic acid tert-butyl ester

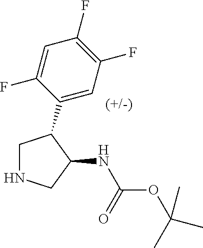

A solution of [(trans)-1-benzyl-4-(2,4,5-trifluorophenyl)pyrrolidin-3-yl]carbamic acid tert-butyl ester (Preparation 54, 40.1 g, 98.8 mmol) in a combination of IMS (325 mL) and EtOAc (50 mL), in an autoclave, was placed under an atmosphere of argon. Palladium on carbon (5%, 4.0 g, 1.9 mmol) was added as a slurry in the minimum volume of toluene, then the reaction mixture was placed under an atmosphere of hydrogen (50 atm) and stirred for 72 h. The crude mixture was filtered through celite, washing with EtOAc, and the filtrate was concentrated in vacuo to afford the title compound. LCMS method 4: RT=2.42 min; ink (ES⁺)=317.2 [M+H]⁺.

### Preparation 56

### [(3R,4S)-4-(2,4,5-Trifluorophenyl)pyrrolidin-3-yl]carbamic acid tert-butyl ester

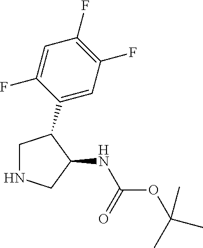

[(trans)-4-(2,4,5-Trifluorophenyl)pyrrolidin-3-yl]carbamic acid tert-butyl ester (Preparation 55, 59.5 g, 188 mmol) was suspended in EtOH (200 mL) and heated to 70° C. To the suspension was added a warm solution of (S)-(+)-naproxen (21.5 g, 93 mmol) and the mixture was heated to reflux. The heat was removed and the mixture slowly allowed to cool to r.t., with stirring, for 16 h. The resulting precipitate was filtered, washing with EtOH, and the filtrate was partitioned between DCM (2400 mL) and 1M NaOH (600 mL). The organic phase was separated, washed with 1M NaOH, brine, then dried (MgSO₄), and the solvent was removed in vacuo. The whole process was repeated for a second time to afford the title compound: ¹H NMR δ_{H} (400 MHz, CD₃OD): 7.38-7.25 (m, 1H), 7.14-7.01 (m, 1H).

### Preparation 57

### 3-Hydroxypyrrolidine-1-carboxylic acid isopropyl ester

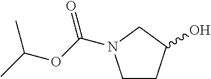

A solution of 3-hydroxypyrrolidine-1-carboxylic acid tert-butyl ester (10.0 g, 53.4 mmol) in a solution of HCl in dioxane (4M, 125 mL) was stirred at r.t. for 40 min before removal of the reaction solvent in vacuo. The residue was suspended in DCM (150 mL), and triethylamine (22.0 mL, 160.2 mmol) was added before cooling the reaction mixture to 0° C. A solution of isopropyl chloroformate in toluene (1M, 64 mL, 64 mmol) was added, dropwise, then the resulting mixture was removed from the ice bath and stirred at r.t. for 1 h. A further portion of isopropyl chloroformate (53.4 ml, 53.4 mmol) was added and stirring continued at r.t. for 16 h. The crude reaction mixture was washed with water (2×100 mL), then dried (MgSO₄). Removal of the solvent in vacuo and purification by column chromatography (DCM:MeOH:NH₄OH, 9:1:0.1) afforded the title compound: RT=2.29 min; m/z (ES⁺)=174.0 [M+H]⁺.

### Preparation 58

### 3-((R)-1-Carboxyethoxy)pyrrolidine-1-carboxylic acid isopropyl ester

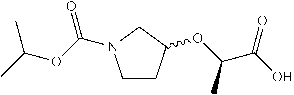

The title compound was prepared employing a similar method to that outlined in Preparation 9. After initial work-up the residue was dissolved in 1M NaOH solution, and washed with Et₂O. The aqueous phase was acidified to pH1 with 2M HCl, extracted with EtOAc, and the organic phase dried (Na₂SO₄). Removal of the solvent in vacuo afforded the title compound: RT=2.70 min; m/z (ES⁺)=246.2 [M+H]⁺.

### Preparation 59

### 3-((R)-1-Carbamoylethoxy)pyrrolidine-1-carboxylic acid isopropyl ester

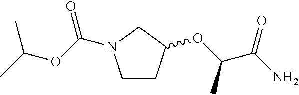

To a solution of 3-((R)-1-carboxyethoxy)pyrrolidine-1-carboxylic acid isopropyl ester (Preparation 58, 1.00 g, 4.08 mmol) in THF (50 mL), under argon, was added EDCI (0.94 g, 4.90 mmol), followed by HOBt (0.66 g, 4.33 mmol), and the reaction was stirred at r.t. for 10 min. A solution of NH₃ in dioxane (0.05M, 20.4 μL, 40.80 mmol) was added and the reaction was stirred at r.t. for 4 h. A further portion of NH₃ in dioxane (0.05M, 10 mL) was added and the reaction was stirred for 1 h. The solvent was removed in vacuo and the resulting residue was partitioned between EtOAc (150 mL) and water (100 mL). The organic phase was removed, and the aqueous phase was extracted with EtOAc (150 mL). The organic fractions were combined, washed with sat. NaHCO₃ solution (100 mL), then brine (100 mL), and dried (MgSO₄). Removal of the solvent in vacuo and purification by column chromatography (DCM:MeOH, 95:5) afforded the title compound: RT=2.53 min, m/z (ES⁺)=245.2 [M+H]⁺.

### Preparation 60

### 3-((R)-Cyanomethylmethoxy)pyrrolidine-1-carboxylic acid isopropyl ester

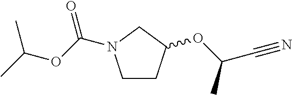

The title compound was prepared from 3-((R)-1-carbamoylethoxy)pyrrolidine-1-carboxylic acid isopropyl ester (Preparation 59) employing the procedure outlined in Preparation 11: RT=3.06 min, m/z (ES⁺)=227.1 [M+H]⁺.

### Preparation 61

### 3-[(R)-1-(N-Hydroxycarbamimidoyl)ethoxy]pyrrolidine-1-carboxylic acid isopropyl ester

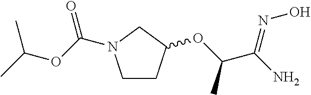

The title compound was prepared from 3-((R)-cyanomethylmethoxy)pyrrolidine-1-carboxylic acid isopropyl ester (Preparation 60) employing the procedure outlined in Preparation 12, but heating the reaction for 2 h: RT=1.99 min, m/z (ES⁺)=260.1 [M+H]⁺.

### Preparation 62

### 3-{(R)-1-[5-(2-Chloropyrimidin-5-yl)-[1,2,4]oxadiazol-3-yl]ethoxy}-pyrrolidine-1-carboxylic acid isopropyl ester

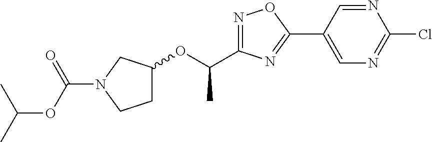

To a solution of 2-chloropyrimidin-2-ol (103 mg, 0.65 mmol) in THF (10 mL), cooled to 0° C., was added 1,3-diisopropylcarbodiimide (101 μL, 0.65 mmol) and the reaction was stirred for 10 min at this temperature. 3-[(R)-1-(N-Hydroxycarbamimidoyl)ethoxy]pyrrolidine-1-carboxylic acid isopropyl ester (Preparation 61, 169 mg, 0.65 mmol) was added, the ice bath was removed, and the mixture was allowed to stir at r.t. for 1 h. The reaction solvent was concentrated to dryness, then the resulting residue was dissolved in EtOAc, washed with water, brine, then dried (Na₂SO₄), and the solvent removed in vacuo. The residue was dissolved in toluene and heated to 80° C. for 18 h before being heated to reflux for 1 h. Removal of the solvent in vacuo and purification by column chromatography (IH:EtOAc, 1:1) afforded the title compound: RT=3.53 min, m/z (ES⁺)=382.1 [M+H]⁺.

### Preparation 63

### 1-Piperidin-4-yl ethanol

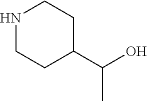

To a solution of α-methyl-4-pyridine methanol (3.7 g, 30 mmol) in EtOH (100 mL) was added AcOH (1.9 mL, 33 mmol) and platinum oxide (0.5 g, 2.2 mmol) and the resulting mixture was allowed to stir under an atmosphere of hydrogen at r.t for 16 h. The mixture was filtered and the filtrate was concentrated in vacuo. The residue was dissolved in MeOH, to which was added a solution of NaOH (1.6 g, 40 mmol) and water (1.6 mL) in MeOH. The reaction was stirred for 30 min before removing the solvent in vacuo, and the resulting residue was suspended in diethyl ether for 30 min. The mixture was filtered and the filtrate was concentrated in vacuo to afford the title compound: ¹H NMR δ_{H} (400 MHz, CDCl₃): 3.63-3.55 (m, 1H), 3.39-3.31 (m, 2H), 2.7-2.6 (m, 2H), 2.01-1.92 (m, 2H), 1.76-1.69 (m, OH), 1.67-1.54 (m, 2H), 1.51-1.42 (m, 1H), 1.1-1.14 (m, 3H).

### Preparation 64

### 4-(1-Hydroxyethyl)piperidine-1-carboxylic acid isopropyl ester

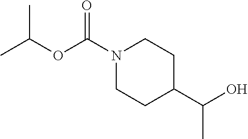

To a solution 1-piperidin-4-yl ethanol (Preparation 63, 5.0 g, 38.76 mmol) in DCM (200 mL) in a 3-necked flask under argon, was added DIPEA (8.8 mL, 50.39 mmol) and the reaction was cooled to 0° C. A solution of isopropylchloroformate in toluene (46.5 mL, 46.5 mmol) was added dropwise, over 10 min, then the reaction was brought to r.t. and stirred for a further 2.5 h. The reaction mixture was diluted with DCM and partitioned with 1M HCl solution. The organic layer was separated, washed with 1M HCl solution, brine, and passed through a phase separator. Removal of the solvent in vacuo afforded the title compound: ¹H NMR 3 (400 MHz, CDCl₃): 4.97-4.87 (m, 1H), 4.28-4.14 (m, 2H), 3.66-3.55 (m, 1H), 2.77-2.63 (m, 2H), 1.88-1.81 (m, 1H), 1.67-1.59 (m, 1H), 1.48-1.38 (m, 1H), 1.26-1.16 (m, 11H).

### Preparation 65

### 4-[1-(5-Bromopyridin-2-yloxy)ethyl]piperidine-1-carboxylic acid isopropyl ester

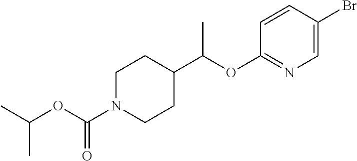

A dry solution of 4-(1-hydroxyethyl)piperidine-1-carboxylic acid isopropyl ester (Preparation 64, 7.4 g, 34.4 mmol) in DMF (100 mL), under argon, was cooled to 0° C. Sodium hydride (60% in mineral oil, 2.8 g, 68.8 mmol) was added in one portion, then the reaction was allowed to stir at r.t. for 1 h. 5-bromo-2-chloropyridine (13.2 g, 68.8 mmol) was added and the reaction was heated to 80° C. for 16 h. The reaction mixture was allowed to cool to r.t. and partitioned between EtOAc and water. The organic phase was separated, washed with water, then brine, and dried (MgSO₄). Removal of the solvent in vacuo, followed by trituration from iso-hexane (2×6 mL) then diethyl ether, afforded the title compound: RT=4.34 min; ink (ES⁺)=371.2 [M+H]⁺.

### Preparation 66

### 4-{1-[5-(4,4,5,5-Tetramethyl-[1,3,2]dioxaborolan-2-yl)pyridin-2-yloxy]ethyl}piperidine-1-carboxylic acid isopropyl ester

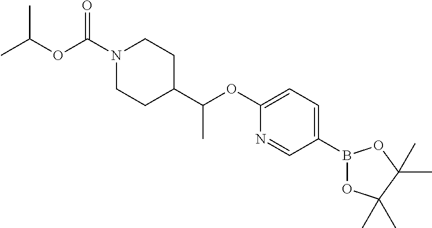

To a solution of 4-[1-(5-bromopyridin-2-yloxy)ethyl]piperidine-1-carboxylic acid isopropyl ester (Preparation 65, 4.2 g, 11.3 mmol) in dioxane (90 mL) was added Potassium acetate (3.3 g, 33.9 mmol), [1,1-bis(diphenylphosphino)ferrocene]dichloropalladium (0.9 g, 1.1 mmol) and bis(pinacolato)diboron (3.4 g, 13.4 mmol). The reaction mixture was bubbled with argon for 10 min, before being heated to 110° C. for 16 h. Removal of the solvent in vacuo followed by purification of the crude material by column chromatography (IH:EtOAc, 100:0, 97.5:2.5, 95:5) afforded the title compound: RT=4.55 min; m/z (ES⁺)=419.4 [M+H]⁺.

### Preparation 67

### (3S,4S)-4-Azido-1-benzylpyrrolidin-3-ylamine

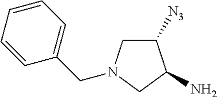

To a solution of (3S,4S)-3,4-diazido-1-benzylpyrrolidine (15.6 g, 64.10 mmol) in THF (500 mL) cooled to 0° C. was added a solution of triphenylphosphine (16.5 g, 62.81 mmol) in THF (100 mL), dropwise over 4 h and the resulting mixture was allowed to reach r.t. and stirred for 16 h. The reaction solvent was removed in vacuo and the resulting residue was re-dissolved in THF (500 mL) and water (1.3 mL) before being heated to reflux for 4 h then stirred at r.t. for 16 h. The reaction solvent was removed in vacuo and the resulting residue was triturated with Et₂O. The precipitate was filtered and the filtrate was concentrated in vacuo. The residue was taken into Et₂O again and filtered. Removal of the filtrate in vacuo followed by purification by column chromatography (IH:EtOAc, 90:10, 80:20, 50:50, 0:100 then MeOH:NH₄OH, 9:1) afforded the title compound: RT=0.77 min; m/z (ES⁺)=218.1 [M+H]⁺.

### Preparation 68

### ((3S,4S)-4-Azido-1-benzylpyrrolidin-3-yl)carbamic acid tert-butyl ester

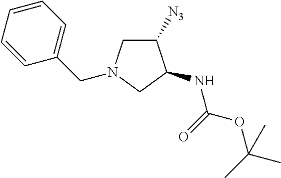

To a solution of (3S,4S)-4-azido-1-benzylpyrrolidin-3-ylamine (Preparation 67, 6.0 g, 27.74 mmol) and triethylamine (4.6 mL, 33.29 mmol) in DCM (100 mL), cooled to 0° C., was added a solution of di tert-butyldicarbonate (7.3 g, 33.29 mmol) in DCM (10 mL) dropwise over 20 min. The resulting mixture was allowed to reach r.t. and stirred for 72 h. The reaction solvent was washed with sat. NaHCO₃ solution, then brine, and dried (MgSO₄). Removal of the solvent in vacuo followed by purification by column chromatography (DCM:MeOH) afforded the title compound: ¹H NMR δ_{H} (400 MHz, CDCl₃): 7.37-7.26 (m, 5H), 4.09-4.02 (m, 1H), 3.84-3.76 (m, 1H), 3.68-3.59 (m, 2H), 3.12-3.01 (m, 1H), 2.91-2.82 (m, 1H), 2.55-2.35 (m, 2H), 1.46 (s, 9H).

### Preparation 69

### ((3S,4S)-4-Amino-1-benzylpyrrolidin-3-yl)carbamic acid tert-butyl ester

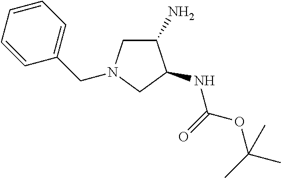

The title compound was prepared from ((3S,4S)-4-azido-1-benzylpyrrolidin-3-yl)carbamic acid tert-butyl ester (Preparation 68) employing the procedure outlined in WO2007/148185.

### Preparation 70

### [(3S,4S)-1-Benzyl-4-(2-oxopiperidin-1-yl)pyrrolidin-3-yl]carbamic acid tert-butyl ester

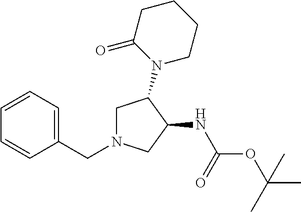

The title compound was prepared in 2 steps from ((3S,4S)-4-amino-1-benzylpyrrolidin-3-yl)carbamic acid tert-butyl ester (Preparation 69) employing the procedures outlined in WO2007/148185.

### Preparation 71

### [(3S,4S)-4-(2-Oxopiperidin-1-yl)pyrrolidin-3-yl]carbamic acid tert-butyl ester

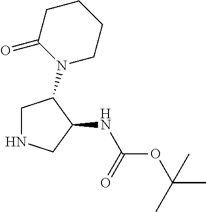

A solution of [(3S,4S)-1-benzyl-4-(2-oxopiperidin-1-yl)pyrrolidin-3-yl]carbamic acid tert-butyl ester (Preparation 70, 2.6 g, 7.07 mmol) in MeOH (140 mL) was passed through an H-Cube apparatus (10% pd/C Catcart 70, 10 bar, 90° C.) at a flow rate of 1 mL per min. The solvent was removed in vacuo to afford the title compound: ¹H NMR δ_{H} (400 MHz, CDCl₃): 5.25-5.07 (m, 1H), 4.85-4.62 (m, 1H), 4.34-4.07 (m, 1H), 3.49-3.28 (m, 3H), 3.24 (s, 1H), 2.99 (s, 1H), 2.87-2.73 (m, 1H), 2.52-2.39 (m, 2H), 2.38-2.22 (m, 2H), 1.91-1.74 (m, 1H), 1.54-1.38 (m, 9H).

### Preparation 72

### [(3S,4S)-1-(5-Bromopyrimidin-2-yl)-4-(2-oxopiperidin-1-yl)pyrrolidin-3-yl]carbamic acid tert-butyl ester

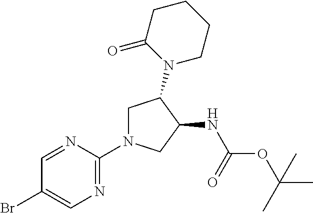

A combination of [(3S,4S)-4-(2-oxopiperidin-1-yl)pyrrolidin-3-yl]carbamic acid tert-butyl ester (Preparation 71, 415 mg, 1.46 mmol), 5-bromo-2-chloropyrimidine (283 mg, 1.46 mmol) and DBU (219 μL, 1.46 mmol) in DMSO (3 mL) was heated to 70° C. for 1 h. The reaction was allowed to cool to room temperature before being partitioned between EtOAc and water. The organic phase was separated, washed with brine, and dried (Na₂SO₄). Removal of the solvent in vacuo, and purification by column chromatography (IH:EtOAc, 1:1) afforded the title compound: RT=3.44 min, m/z (ES⁺)=440.1 [M+H]⁺.

### Preparation 73

### [(3R,4S)-1-(5-Bromopyrimidin-2-yl)-4-(2,5-difluorophenyl)pyrrolidin-3-yl]carbamic acid tert-butyl ester

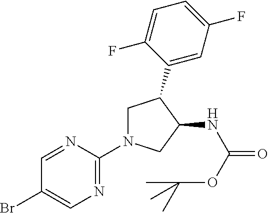

A combination of [(3R,4S)-4-(2,5-difluorophenyl)pyrrolidin-3-yl]carbamic acid tert-butyl ester (Preparation 48, 540 mg, 1.8 mmol), 5-bromo-2-chloropyrimidine (350 mg, 1.8 mmol) and DBU (270 μL, 1.8 mmol) in DMSO (3 mL) was heated to 70° C. for 30 min. The reaction was allowed to cool to room temperature before being partitioned between EtOAc and water. The organic phase was separated, washed with brine, and dried (Na₂SO₄). Removal of the solvent in vacuo and purification by column chromatography (IH:EtOAc, 1:1) afforded the title compound: RT=4.32 min, m/z (ES⁺)=455.1 [M+H]⁺.

### Example 1

### 4-(5-{6-[(3R,4S)-3-Amino-4-(2-fluorophenyl)pyrrolidin-1-yl]pyridine-3-yl}-[1,2,4]oxadiazol-3-ylmethoxy)piperidine-1-carboxylic acid isopropyl ester

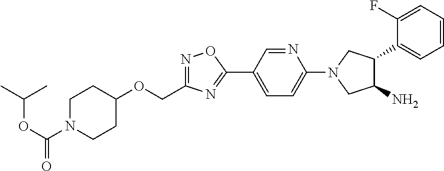

A combination of 4-[5-(6-chloropyridin-3-yl)-[1,2,4]oxadiazol-3-ylmethoxy]piperidine-1-carboxylic acid isopropyl ester (Preparation 5, 150 mg, 0.39 mmol), [(trans)-4-(2-fluorophenyl)pyrrolidin-3-yl]carbamic acid-9H-fluoren-9-ylmethyl ester hydrochloride (Preparation 30), 172 mg, 0.39 mmol) and DIPEA (143 μL, 0.82 mmol) in tert-butanol (2 mL) was heated to 80° C. until the reaction was complete. The mixture was diluted with a small volume of DCM and purified by column chromatography (DCM:MeOH, 96:4). Further purification by chiral HPLC (MTBE:EtOH:BA 80:20:0.1, 11 mL/min, 285 nm, RT=35.0 min) afforded the title compound: RT=2.75 min; m/z (ES⁺)=525.2 [M+H]⁺.

### Example 2

### 4-(5-{2-[(3R,4S)-3-Amino-4-(2-fluorophenyl)pyrrolidin-1-yl]pyrimidin-5-yl}-[1,2,4]oxadiazol-3-ylmethoxy)piperidine-1-carboxylic acid isopropyl ester p-toluenesulfonic acid salt

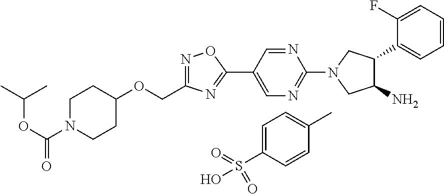

A combination of 4-[5-(6-chloropyrimidin-5-yl)-[1,2,4]oxadiazol-3-ylmethoxy]piperidine-1-carboxylic acid isopropyl ester (Preparation 6, 150 mg, 0.39 mmol), [(trans)-4-(2-fluorophenyl)pyrrolidin-3-yl]carbamic acid-9H-fluoren-9-ylmethyl ester hydrochloride (Preparation 30), 172 mg, 0.39 mmol) and DIPEA (143 μL, 0.83 mmol) in tert-butanol (2 mL) was heated to 80° C. for 16 h. The reaction mixture was concentrated in vacuo and re-dissolved in DCM (150 mL). The organic mixture was washed with water (50 mL), brine (50 mL), then dried (MgSO₄), and the solvent was removed in vacuo. Purification by column chromatography (DCM:MeOH, 96:4) followed by chiral HPLC (MeOH:THF:CHCl₃ 55:25:20, 11 ml/min, 285 nm, RT=6.8 min) afforded the intermediate product 4-(5-{2-[(3R,4S)-3-(9H-fluoren-9-ylmethoxycarbonylamino)-4-(2-fluorophenyl)pyrrolidin-1-yl]pyrimidin-5-yl}-[1,2,4]oxadiazol-3-ylmethoxy)piperidine-1-carboxylic acid isopropyl ester: RT=4.89 min; m/z (ES⁺)=748.3 [M+H]⁺. To a solution of the product in DCM (1 mL), cooled to 0° C., was added a solution of piperidine (0.5 mL) in DCM (1 mL), dropwise. The mixture was stirred at this temperature for 2 h before removal of the solvent in vacuo and purification by column chromatography (DCM:MeOH, 98:2, 92:8). A solution of TsOH (1 eq.) in MeOH (2 mL) was added to the product before removal of the solvent in vacuo to afford the title compound: RT=2.78 min; m/z (ES⁺)=526.4 [M+H]⁺.

### Example 3

### 4-(5-{2-[(3R,4S)-3-Amino-4-(2,5-difluorophenyl)pyrrolidin-1-yl]pyrimidin-5-yl}-[1,2,4]oxadiazol-3-ylmethoxy)piperidine-1-carboxylic acid isopropyl ester p-toluenesulfonic acid salt

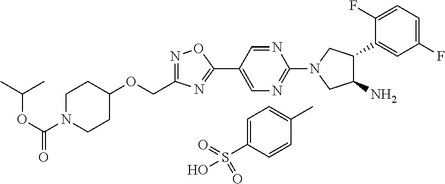

A combination of 4-[5-(6-chloropyrimidin-5-yl)-[1,2,4]oxadiazol-3-ylmethoxy]piperidine-1-carboxylic acid isopropyl ester (Preparation 6, 65 mg, 0.17 mmol), [(3R,4S)-4-(2,5-difluorophenyl)pyrrolidin-3-yl]carbamic acid tert-butyl ester (Preparation 48, 59 mg, 0.17 mmol) and DIPEA (38 μL, 0.22 mmol) in tert-butanol (1 mL) was heated to 80° C. until the reaction was complete. The reaction mixture was concentrated in vacuo and purified by column chromatography (DCM:MeOH, 99:1, 96:4) to afford the intermediate product 4-(5-{2-[(3R,4S)-3-tert-butoxycarbonylamino-4-(2,5-difluorophenyl)pyrrolidin-1-yl]pyrimidin-5-yl}-[1,2,4]oxadiazol-3-ylmethoxy)piperidine-1-carboxylic acid isopropyl ester: RT=4.47 min; m/z (ES⁺)=644.5 [M+H]⁺. To a solution of the product in DCM (5 mL), cooled to 0° C., was added TFA (1 mL), dropwise, and the reaction was stirred at r.t. for 45 min. The mixture was diluted with DCM (75 mL), washed with sat. Na₂CO₃ solution (25 mL), then dried (MgSO₄), and the solvent was removed in vacuo. Purification by column chromatography (DCM:MeOH, 96:4) afforded the title compound as the free amine. A solution of TsOH (1 eq.) in MeOH (2 mL) was added to the product and the solvent was removed in vacuo to afford the title compound: RT=2.79 min; m/z (ES⁺)=544.2 [M+H]⁺.

### Example 4

### 4-(5-{2-[(3R,4S)-3-Amino-4-(2,4-difluorophenyl)pyrrolidin-1-yl]pyrimidin-5-yl}-[1,2,4]oxadiazol-3-ylmethoxy)piperidine-1-carboxylic acid isopropyl ester p-toluenesulfonic acid salt

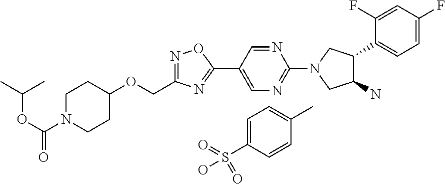

The title compound was prepared from 4-[5-(6-chloropyrimidin-5-yl)-[1,2,4]oxadiazol-3-ylmethoxy]piperidine-1-carboxylic acid isopropyl ester (Preparation 6) and [(3R,4S)-4-(2,4-difluorophenyl)pyrrolidin-3-yl]carbamic acid tert-butyl ester (Preparation 37) employing the procedure outlined in Example 3: RT=2.75 min; m/z (ES⁺)=544.3 [M+H]⁺.

### Example 5

### 4-(3-{6-[(3R,4S)-3-Amino-4-(2-fluorophenyl)pyrrolidin-1-yl]pyridine-3-yl}-[1,2,4]oxadiazol-5-ylmethoxy)piperidine-1-carboxylic acid isopropyl ester p-toluenesulfonic acid salt

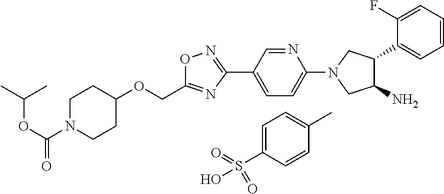

4-[3-(6-Chloropyridin-3-yl)-[1,2,4]oxadiazol-5-ylmethoxy]piperidine-1-carboxylic acid isopropyl ester (Preparation 7) was reacted with [(trans)-4-(2-fluorophenyl)pyrrolidin-3-yl]carbamic acid-9H-fluoren-9-ylmethyl ester hydrochloride (Preparation 30) employing the procedure outlined in Example 1 to give the title compound as the free amine (chiral HPLC: MTBE:MeOH:BA 80:20:0.1, 12 mL/min, 285 nm, RT=32.8 min) A solution of TsOH (1 eq.) in MeOH (5 mL) was added to the product and the solvent was removed in vacuo to afford the title compound: RT=2.80 min; m/z (ES⁺)=525.2 [M+H]⁺.

### Example 6

### 4-(3-{6-[(3S,4R)-3-Amino-4-(2-fluorophenyl)pyrrolidin-1-yl]pyridine-3-yl}-[1,2,4]oxadiazol-5-ylmethoxy)piperidine-1-carboxylic acid isopropyl ester p-toluenesulfonic acid salt

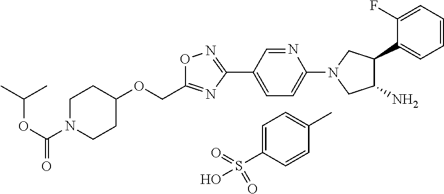

The title compound was prepared from 4-[3-(6-chloropyridin-3-yl)-[1,2,4]oxadiazol-5-ylmethoxy]piperidine-1-carboxylic acid isopropyl ester (Preparation 7) and [(trans)-4-(2-fluorophenyl)pyrrolidin-3-yl]carbamic acid-9H-fluoren-9-ylmethyl ester hydrochloride (Preparation 30), employing the procedure outlined in Example 5. Chiral HPLC: MTBE:MeOH:BA 80:20:0.1, 12 mL/min, 285 nm, RT=29.2 min LCMS: RT=2.80 min, m/z (ES⁺)=525.2 [M+H]⁺.

### Example 7

### 4-(3-{6-[(3S,4R)-3-Amino-4-(2-fluorophenyl)pyrrolidin-1-yl]pyridine-3-yl}-[1,2,4]oxadiazol-5-ylmethoxy)piperidine-1-carboxylic acid isopropyl ester p-toluenesulfonic acid salt

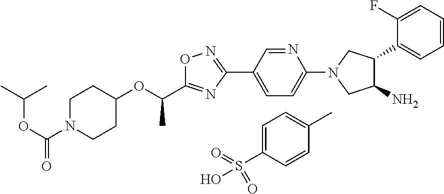

The title compound was prepared from 4-{(R-1-[3-(6-chloropyridin-3-yl)-[1,2,4]oxadiazol-5-yl]ethoxy}piperidine-1-carboxylic acid isopropyl ester (Preparation 8) and [(trans)-4-(2-fluorophenyl)pyrrolidin-3-yl]carbamic acid-9H-fluoren-9-ylmethyl ester hydrochloride (Preparation 30), employing the procedure outlined in Example 5. Chiral HPLC: MTBE:MeOH:BA 80:20:0.1, 12 mL/min, 285 nm RT=20.4 min. LCMS: RT=2.89 min; m/z (ES⁺)=539.5 [M+H]⁺.

### Example 8

### 4-(3-{6-[(3R,4S)-3-Amino-4-(2,5-difluorophenyl)pyrrolidin-1-yl]pyridin-3-yl}-[1,2,4]oxadiazol-5-ylmethoxy)piperidine-1-carboxylic acid isopropyl ester p-toluenesulfonic acid salt

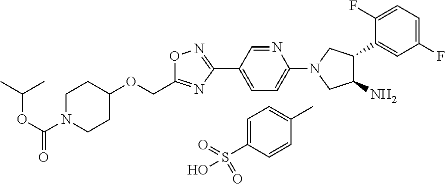

4-[3-(6-Chloropyridin-3-yl)-[1,2,4]oxadiazol-5-ylmethoxy]piperidine-1-carboxylic acid isopropyl ester (Preparation 7) was reacted with [(3R,4S)-4-(2,5-difluorophenyl)pyrrolidin-3-yl]carbamic acid tert-butyl ester (Preparation 48) employing the procedure outlined in Example 3. After reaction with TFA the crude mixture was passed down an SCX cartridge, eluting with MeOH then NH₄OH in MeOH, and the basic fraction was collected to afford the title compound as the free amine. To a solution of the product in MeOH was added TsOH (1 eq.) in MeOH, then the solvent was removed in vacuo to afford the title compound: RT=2.82 min; m/z (ES⁺)=543.2 [M+H]⁺.

### Example 9

### 4-[(R)-1-(3-{6-[(3R,4S)-3-Amino-4-(2,5-difluorophenyl)pyrrolidin-1-yl]pyridin-3-yl}-[1,2,4]oxadiazol-5-yl)ethoxy]piperidine-1-carboxylic acid isopropyl ester p-toluenesulfonic acid salt

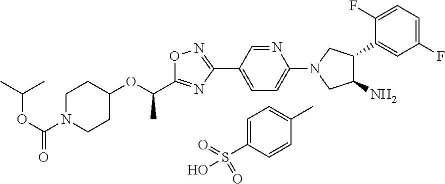

A combination of 4-{(R-1-[3-(6-chloropyridin-3-yl)-[1,2,4]oxadiazol-5-yl]ethoxy}piperidine-1-carboxylic acid isopropyl ester (Preparation 8, 79 mg, 0.20 mmol), [(3R,4S)-4-(2,5-difluorophenyl)pyrrolidin-3-yl]carbamic acid tert-butyl ester (Preparation 48, 60 mg, 0.20 mmol) and DIPEA (38 μL, 0.22 mmol) in tert-butanol (1 mL) was heated to 80° C. for 72 h and then 85° C. until no further reaction. The mixture was concentrated in vacuo and purified by column chromatography (DCM:MeOH, 99:1, 97:3) to afford the intermediate product 4-[(R)-1-(3-{6-[(3R,4S)-3-tert-butoxycarbonylamino-4-(2,5-difluorophenyl)pyrrolidin-1-yl]pyridin-3-yl}-[1,2,4]oxadiazol-5-yl)ethoxy]piperidine-1-carboxylic acid isopropyl ester: RT=4.39 min; m/z (ES⁺)=657.4 [M+H]⁺. To a solution of the product in DCM (5 mL), cooled to 0° C., was added TFA (1 mL), dropwise, and the reaction was stirred at r.t. for 45 min. The mixture was diluted with DCM, washed with sat. Na₂CO₃ solution, dried (MgSO₄), and the solvent removed in vacuo. Purification by column chromatography (DCM:MeOH, 97:3) afforded the title compound as the free amine. A solution of TsOH (1 eq.) in MeOH (2 mL) was added to the product, then the solvent was removed in vacuo to afford the title compound: RT=2.93 min; m/z (ES⁺)=557.3 [M+H]⁺.

### Example 10

### 4-[(R)-1-(5-{2-[(3R,4S)-3-Amino-4-(2,5-difluorophenyl)pyrrolidin-1-yl]pyrimidin-5-yl}-[1,2,4]oxadiazol-3-yl)ethoxy]piperidine-1-carboxylic acid isopropyl ester

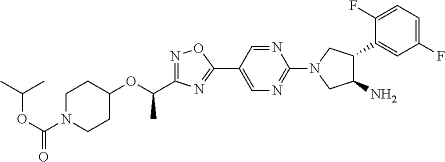

4-{(R)-1-[5-(2-Chloropyrimidin-5-yl)-[1,2,4]oxadiazol-3-yl]ethoxy}piperidine-1-carboxylic acid isopropyl ester (Preparation 13) was reacted with [(3R,4S)-4-(2,5-difluorophenyl)pyrrolidin-3-yl]carbamic acid tert-butyl ester (Preparation 48) employing the procedure outlined in Example 3. After reaction with TFA the crude mixture was passed down an SCX cartridge, eluting with MeOH then NH₄OH in MeOH, and the basic fraction was collected to afford the title compound: RT=2.80 min; m/z (ES⁺)=558.2 [M+H]⁺.

### Example 11

### 4-[(R)-1-(5-{5-[(3R,4S)-3-Amino-4-(2,5-difluorophenyl)pyrrolidin-1-yl]pyrazin-2-yl}-[1,2,4]oxadiazol-3-yl)ethoxy]piperidine-1-carboxylic acid isopropyl ester

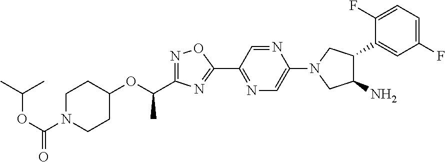

To a solution of 4-{(R)-1-[5-(5-chloropyrazin-2-yl)-[1,2,4]oxadiazol-3-yl]ethoxy}piperidine-1-carboxylic acid isopropyl ester (Preparation 14, 127 mg, 0.32 mmol) in DMSO (0.5 mL) was added [(3R,4S)-4-(2,5-difluorophenyl)pyrrolidin-3-yl]carbamic acid tert-butyl ester (Preparation 48, 98 mg, 0.33 mmol) and DBU (48 μL, 0.32 mmol), and the reaction was heated to 70° C. for 1 h. The mixture was partitioned between DCM (100 mL) and water (50 mL), and the organic phase was separated. The aqueous phase was extracted with DCM (50 mL), then the organic fractions were combined, washed with brine, and dried (MgSO₄). Purification by column chromatography (DCM:MeOH, 98:2) afforded the intermediate product 4-[(R)-1-(5-{5-[(3R,4S)-3-tert-butoxycarbonylamino-4-(2,5-difluorophenyl)pyrrolidin-1-yl]pyrazin-2-yl}-[1,2,4]oxadiazol-3-yl)ethoxy]piperidine-1-carboxylic acid isopropyl ester: RT=4.43 min; m/z (ES⁺)=658.3 [M+H]⁺. To a solution of the product in DCM (5 mL), cooled to 0° C., was added TFA (1 mL), dropwise, and the reaction was stirred at r.t. until complete. Removal of the solvent in vacuo and purification by column chromatography (DCM:MeOH, 95:5) afforded the title compound: RT=2.74 min; m/z (ES⁺)=558.2 [M+H]⁺.

### Example 12

### 4-{5-[(3R,4R)-3-Amino-4-(2,5-difluorophenyl)-3,4,5,6-tetrahydro-2H-[1,2]bipyridinyl-5′yl]-[1,2,4]oxadiazol-3-ylmethoxy}piperidine-1-carboxylic acid isopropyl ester hydrochloride

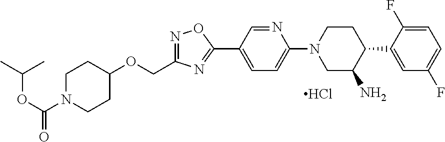

A combination of 4-[5-(6-chloropyridin-3-yl)-[1,2,4]oxadiazol-3-ylmethoxy]piperidine-1-carboxylic acid isopropyl ester (Preparation 5, 38 mg, 0.10 mmol), [(3R,4R)-4-(2,5-difluorophenyl)piperidin-3-yl]carbamic acid tert-butyl ester (Preparation 19, 31 mg, 0.10 mmol), and DIPEA (18 μL, 0.11 mmol) in tert-butanol (0.30 mL) was heated to 80° C. until the reaction was complete. The mixture was diluted with EtOAc (50 mL), washed with brine (50 mL), dried (MgSO₄), and concentrated in vacuo to afford the intermediate product 4-{5-[(3R,4R)-3-tert-butylcarbonylamino-4-(2,5-difluorophenyl)-3,4,5,6-tetrahydro-2H-[1,2′]bipyridinyl-5′yl]-[1,2,4]oxadiazol-3-ylmethoxy}piperidine-1-carboxylic acid isopropyl ester: RT=4.53 min; m/z (ES⁺)=657.2 [M+H]⁺. To a solution of the product in DCM (3 mL) was added TFA (3 mL) and the reaction was stirred at r.t. for 30 min. The crude mixture was passed down an SCX cartridge, eluting with MeOH then NH₄OH in MeOH, and the basic fraction was collected and concentrated in vacuo to afford the title compound as the free amine. The residue was dissolved in a solution of HCl in dioxane (4M), then the solvent was removed in vacuo to afford the title compound: RT=2.85 min; m/z (ES⁺)=557.3 [M+H]⁺.

### Example 13

### 4-{3-[(3R,4R)-3-Amino-4-(2,5-difluorophenyl)-3,4,5,6-tetrahydro-2H-[1,2]bipyridinyl-5′-yl]-[1,2,4]oxadiazol-5-ylmethoxy}piperidine-1-carboxylic acid isopropyl ester dihydrochloride

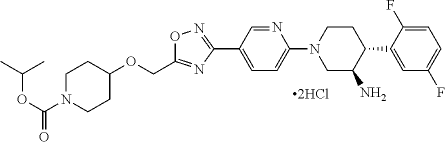

The title compound was prepared from 4-[3-(6-chloropyridin-3-yl)-[1,2,4]oxadiazol-5-ylmethoxy]piperidine-1-carboxylic acid isopropyl ester (Preparation 7) and [(3R,4R)-4-(2,5-difluorophenyl)piperidin-3-yl]carbamic acid tert-butyl ester (Preparation 19) employing the procedure outlined in Example 12. After purification by SCX cartridge the residue was further purified by preparative HPLC. Salt formation employing the method outlined in Example 12 afforded the title compound as the dihydrochloride salt: RT=2.73 min; m/z (ES⁺)=557.2 [M+H]⁺.

### Example 14

### 4-(3-{2-[(3R,4S)-3-Amino-4-(2,4-difluorophenyl)pyrrolidin-1-yl]pyrimidin-5-yl}-[1,2,4]oxadiazol-5-ylmethoxy)piperidine-1-carboxylic acid isopropyl ester p-toluenesulfonic acid salt

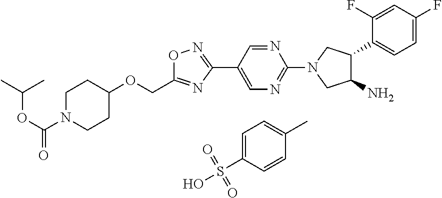

To a solution of 4-[3-(2-chloropyrimidin-5-yl)-[1,2,4]oxadiazol-5-ylmethoxy]piperidine-1-carboxylic acid isopropyl ester (Preparation 25, 40 mg, 0.10 mmol) and [(3R,4S)-4-(2,4-difluorophenyl)pyrrolidin-3-yl]carbamic acid tert-butyl ester (Preparation 37, 38 mg, 0.13 mmol) in DMSO (0.4 mL) was added DBU (16 μL, 0.10 mmol), and the mixture was bubbled with argon for 1 min before being heated to 70° C. for 16 h. The reaction mixture was partitioned between EtOAc (50 mL) and water (20 mL), and the organic phase was separated. The aqueous phase was extracted with EtOAc (20 mL) then organic fractions were combined, washed with sat. NaHCO₃ solution (50 mL), brine (50 mL), then dried (MgSO₄), and the solvent was removed in vacuo. Purification by column chromatography (DCM:MeOH, 100:0, 98:2) afforded the intermediate product 4-(3-{2-[(3R,4S)-3-tert-butoxycarbonylamino-4-(2,4-difluorophenyl)pyrrolidin-1-yl]pyrimidin-5-yl}-[1,2,4]oxadiazol-5-ylmethoxy)piperidine-1-carboxylic acid isopropyl ester: RT=4.43 min; m/z (ES⁺)=644.3 [M+H]⁺. To a solution of the product in DCM (5 mL), under argon, cooled to 0° C., was added TFA (1 mL) and the reaction was stirred at this temperature for 2 h. Further TFA (0.5 mL) was added and stirring continued until the reaction was complete. The crude mixture was passed down an SCX cartridge, eluting with MeOH then NH₄OH in MeOH. The basic fraction was collected and concentrated in vacuo to afford the title compound as the free amine. To a solution of the product in DCM (2 mL) was added a solution of TsOH (1 eq.) in MeOH (2 mL), then the solvent was removed in vacuo to afford the title compound: RT=2.86 min; m/z (ES⁺)=544.2 [M+H]⁺.

The following examples were prepared by reacting the appropriate 2-chloropyrimidine intermediate with the appropriate amine building block employing the procedure employed in Example 14:

| Ex. | Structure | Name | LCMS |
|---|---|---|---|
| 15 | 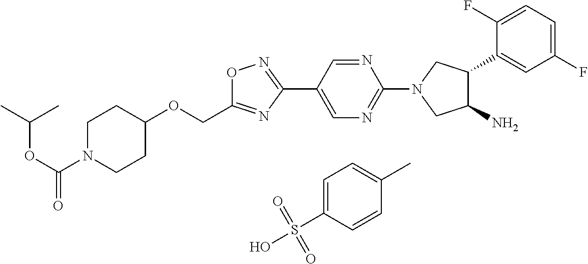 | 4-(3-{2-[(3R,4S)-3- Amino-4-(2,5- difluorophenyl)pyrrolidin- 1-yl]pyrimidin-5-yl}- [1,2,4]oxadiazol-5- ylmethoxy)piperidine-1- carboxylic acid isopropyl ester p-toluenesulfonic acid salt | RT = 2.82 min, m/z (ES⁺) = 544.2 [M + H]⁺ |
| 16 | 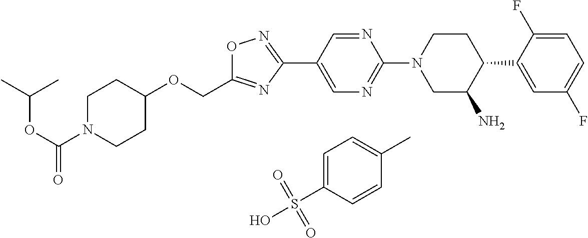 | 4-(3-{2-[(3R,4R)-3- Amino-4-(2,5- difluorophenyl)piperidin- 1-yl]pyrimidin-5-yl}- [1,2,4]oxadiazol-5- ylmethoxy)piperidine-1- carboxylic acid isopropyl ester p-toluenesulfonic acid salt | RT = 2.92 min, m/z (ES⁺) = 558.2 [M + H]⁺ |
| 17 | 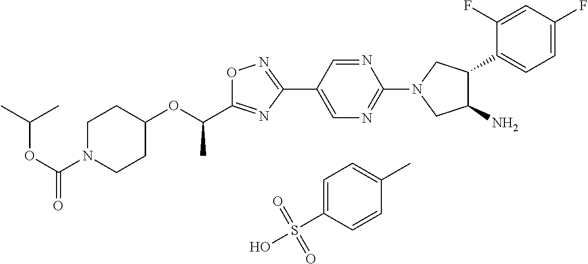 | 4-[(R)-1-(3-{2-[(3R,4S)- 3-Amino-4-(2,4- difluorophenyl)pyrrolidin- 1-yl]pyrimidin-5-yl}- [1,2,4]oxadiazol-5- yl)ethoxy]piperidine-1- carboxylic acid isopropyl ester p-toluenesulfonic acid salt | RT = 2.93 min, m/z (ES⁺) = 558.2 [M + H]⁺ |
| 18 | 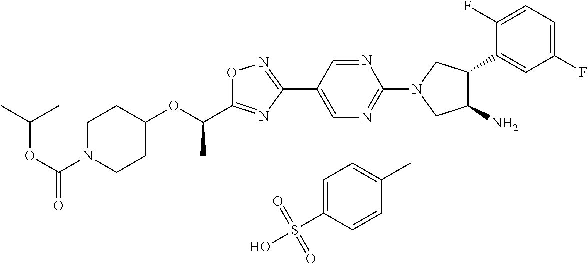 | 4-[(R)-1-(3-{2-[(3R,4S)- 3-Amino-4-(2,5- difluorophenyl)pyrrolidin- 1-yl]pyrimidin-5-yl}- [1,2,4]oxadiazol-5- yl)ethoxy]piperidine-1- carboxylic acid isopropyl ester p-toluenesulfonic acid salt | RT = 2.94 min, m/z (ES⁺) = 558.2 [M + H]⁺ |

### Example 19

### 4-(3-{2-[(3R,4S)-3-Amino-4-(2-fluorophenyl)pyrrolidin-1-yl]pyrimidin-5-yl}-[1,2,4]oxadiazol-5-ylmethoxy)piperidine-1-carboxylic acid isopropyl ester p-toluenesulfonic acid salt

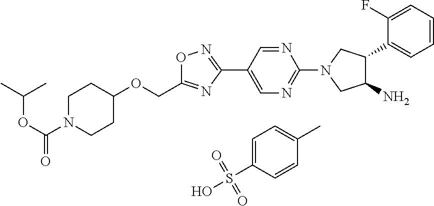

To a solution of 4-[3-(2-chloropyrimidin-5-yl)-[1,2,4]oxadiazol-5-ylmethoxy]piperidine-1-carboxylic acid isopropyl ester (Preparation 25, 19 mg, 0.05 mmol) and [(3R,4S)-4-(2-fluorophenyl)pyrrolidin-3-yl]carbamic acid-9H-fluoren-9-ylmethyl ester hydrochloride (Preparation 32, 31 mg, 0.07 mmol) in DMSO (0.4 mL), under argon, was added DBU (15 μL, 0.10 mmol) and the reaction was heated to 80° C. for 65 h. The mixture was diluted with water (10 mL) and the resulting solution extracted with EtOAc (3×30 mL). The organic fractions were combined, washed with water (30 mL), sat. NaHCO₃ solution (30 mL), and brine (30 mL), then dried (MgSO₄). Removal of the solvent in vacuo and purification by column chromatography (DCM:MeOH, 100:0, 98:2, 97:3, 95:5, 92:8) afforded the title compound as the free amine. To a solution of the product in DCM (4 mL) was added a solution of TsOH (1 eq.) in MeOH (2 mL), then the solvent was removed in vacuo to afford the title compound: RT=2.74 min; m/z (ES⁺)=526.2 [M+H]⁺.

### Example 20

### 4-[(R)-1-(3-{2-[(3R,4S)-3-Amino-4-(2-fluorophenyl)pyrrolidin-1-yl]pyrimidin-5-yl]-[1,2,4]oxadiazol-5-yl)ethoxy}piperidine-1-carboxylic acid isopropyl ester p-toluenesulfonic acid salt

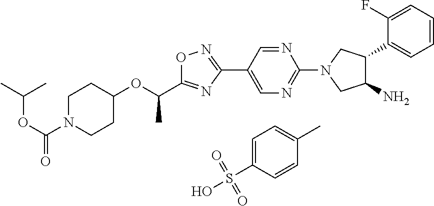

The title compound was prepared by reacting 4-{(R)-1-[3-(2-chloropyrimidin-5-yl)-[1,2,4]oxadiazol-5-yl]ethoxy}piperidine-1-carboxylic acid isopropyl ester (Preparation 28) with [(3R,4S)-4-(2-fluorophenyl)pyrrolidin-3-yl]carbamic acid-9H-fluoren-9-ylmethyl ester hydrochloride (Preparation 32) employing the procedure outlined in Example 19: RT=2.84 min; m/z (ES⁺)=540.3 [M+H]⁺.

### Example 21

### 4-[(R)-1-(5-{6-[(3R,4S)-3-Amino-4-(2,5-difluorophenyl)pyrrolidin-1-yl]pyridazin-3-yl}-[1,2,4]oxadiazol-3-yl)ethoxy]piperidine-1-carboxylic acid isopropyl ester

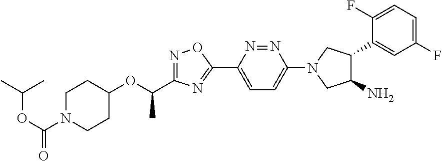

A combination of 4-((R)-1-{5-[6-(benzotriazol-1-yloxy)pyridazin-3-yl]-[1,2,4]oxadiazol-3-yl}ethoxy)piperidine-1-carboxylic acid isopropyl ester (Preparation 38, 12 mg, 0.02 mmol) and [(3R,4S)-4-(2,5-difluorophenyl)pyrrolidin-3-yl]carbamic acid tert-butyl ester (Preparation 48, 8 mg, 0.03 mmol) in DMSO (0.5 mL) was heated to 70° C. for 1 h. The reaction mixture was diluted with water and extracted with EtOAc (2×50 mL). The organic fractions were combined, washed with brine, dried (MgSO₄) and the solvent removed in vacuo to afford the intermediate product 4-[(R)-1-(5-{6-[(3R,4S)-3-tert-butoxycarbonylamino-4-(2,5-difluorophenyl)pyrrolidin-1-yl]pyridazin-3-yl}-[1,2,4]oxadiazol-3-yl)ethoxy]piperidine-1-carboxylic acid isopropyl ester: RT=4.23 min; m/z (ES⁺)=658.3 [M+H]⁺. To a solution of the product in DCM (5 mL), cooled to 0° C., was added TFA (0.5 mL), dropwise, and the reaction was stirred at r.t. for 1 h. The crude mixture was passed down an SCX cartridge, eluting with MeOH then NH₄OH in MeOH, and the basic fraction was collected and concentrated in vacuo. Further purification by column chromatography (DCM:MeOH, 95:5) afforded the title compound: RT=2.72 min; m/z (ES⁺)=558.2 [M+H]⁺.

### Example 22

### 3-[(R)-1-(3-{2-[(3R,4S)-3-Amino-4-(2,5-difluorophenyl)pyrrolidin-1-yl]pyrimidin-5-yl}-[1,2,4]oxadiazol-5-yl)ethoxy]azetidine-1-carboxylic acid isopropyl ester p-toluenesulfonic acid salt

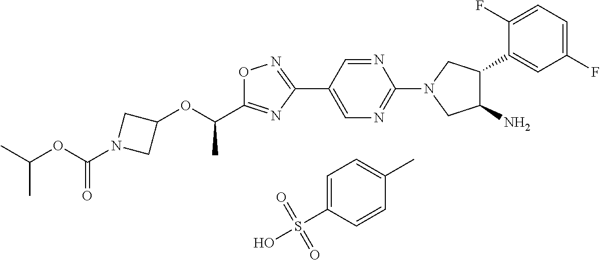

To a solution of 3-[(R)-1-(3-{2-[(3R,4S)-3-tert-butoxycarbonylamino-4-(2,5-difluorophenyl)pyrrolidin-1-yl]pyrimidin-5-yl}-[1,2,4]oxadiazol-5-yl)ethoxy]azetidine-1-carboxylic acid isopropyl ester (Preparation 51,109 mg, 0.17 mmol) in DCM (5 mL), cooled to 0° C., was added TFA (1.0 mL), dropwise, and the reaction was stirred at this temperature for 2 h. A further portion of TFA (0.5 mL) was added and stirring continued for 30 min. The crude reaction mixture was passed down an SCX cartridge, eluting with MeOH then NH₄OH in MeOH, and the basic fraction was collected and concentrated in vacuo. To a solution of the product in DCM (2 mL) was added a solution of TsOH (1 eq.) in MeOH (2 mL), then the solvent was removed in vacuo to afford the title compound: RT=2.73 min; m/z (ES⁺)=530.2 [M+H]⁺.

### Example 23

### 4-[(R)-1-(3-{5-[(3R,4S)-3-Amino-4-(2,5-difluorophenyl)pyrrolidin-1-yl]pyrazin-2-yl}-[1,2,4]oxadiazol-5-yl)ethoxy]piperidine-1-carboxylic acid isopropyl ester

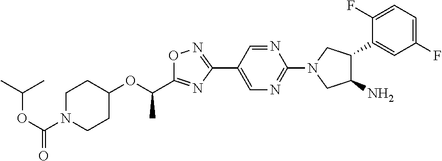

To a solution of 4-((R)-1-carboxyethoxy)piperidine-1-carboxylic acid isopropyl ester (Preparation 9, 72 mg, 0.28 mmol) in THF (20 mL) was added EDCI (63 mg, 0.33 mmol), followed by HOBt (45 mg, 0.30 mmol), and the reaction was stirred at r.t. for 10 min. {(3R,4S)-4-(2,5-Difluorophenyl)-1-[5-(N-hydroxycarbamimidoyl)pyrazin-2-yl]pyrrolidin-3-yl}carbamic acid tert-butyl ester (Preparation 53, 120 mg, 0.28 mmol) was added and the reaction was stirred at r.t. for 16 h. The reaction solvent was concentrated in vacuo and the resulting residue was partitioned between EtOAc (100 mL) and water (50 mL). The organic phase was separated, dried (MgSO₄), and concentrated in vacuo. The residue was taken into toluene and heated to reflux for 16 h. Removal of the solvent in vacuo and purification by column chromatography (DCM:MeOH, 97:3) afforded the intermediate product: RT=4.39 min; m/z (ES⁺)=658.3 [M+H]⁺. To a solution of the product in DCM (7 mL), cooled to 0° C., was added TFA (0.7 mL). The ice bath was removed and the reaction was stirred at r.t. for 1 h. Purification by preparative HPLC followed by column chromatography (DCM:MeOH, 95:5) afforded the title compound: RT=2.79 min; m/z (ES⁺)=5581 [M+H]⁺.

### Example 24

### 4-[(R)-1-(5-{2-[(3R,4S)-3-Amino-4-(2,4,5-trifluorophenyl)pyrrolidin-1-yl]pyrimidin-5-yl}-[1,2,4]oxadiazol-3-yl)ethoxy]piperidine-1-carboxylic acid isopropyl ester

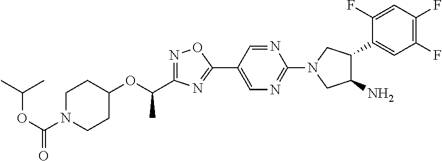

A combination of 4-{(R)-1-[5-(2-chloropyrimidin-5-yl)-[1,2,4]oxadiazol-3-yl]ethoxy}piperidine-1-carboxylic acid isopropyl ester (Preparation 13, 100 mg, 0.25 mmol) and [(3R,4S)-4-(2,4,5-trifluorophenyl)pyrrolidin-3-yl]carbamic acid tert-butyl ester (Preparation 56, 80 mg, 0.25 mmol) in DMSO (1.0 mL) was treated with DBU (38 μL, 0.25 mmol) and the reaction was heated to 70° C. for 3 h. The reaction mixture was diluted with water (70 mL) and extracted with EtOAc (2×100 mL). The organic fractions were combined, washed with brine, dried (MgSO₄) and the solvent was removed in vacuo to afford the intermediate product 4-[(R)-1-(5-{2-[(3R,4S)-3-tert-butoxycarbonylamino-4-(2,4,5-trifluorophenyl)pyrrolidin-1-yl]pyrimidin-5-yl}-[1,2,4]oxadiazol-3-yl)ethoxy]piperidine-1-carboxylic acid isopropyl ester: RT=4.69 min; m/z (ES⁺)=676.3 [M+H]⁺. To a solution of the product in DCM (7 mL), cooled to 0° C., was added TFA (0.7 mL), dropwise, and the resulting reaction was stirred at r.t. for 1 h. The crude mixture was passed down an SCX cartridge, eluting with MeOH then NH₄OH in MeOH, and the basic fraction was collected and concentrated in vacuo to afford the title compound: RT=2.88 min; m/z (ES⁺)=576.2 [M+H]⁺.

### Example 25

### (R)-3-[(R)-1-(5-{2-[(3R,4S)-3-Amino-4-(2,5-difluorophenyl)pyrrolidin-1-yl]pyrimidin-5-yl}-[1,2,4]oxadiazol-3-yl)ethoxy]pyrrolidine-1-carboxylic acid isopropyl ester

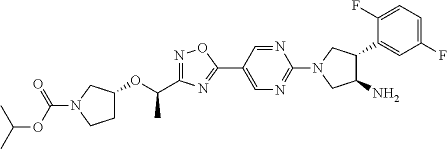

A combination of 3-{(R)-1-[5-(2-chloropyrimidin-5-yl)-[1,2,4]oxadiazol-3-yl]ethoxy}pyrrolidine-1-carboxylic acid isopropyl ester (Preparation 62, 60 mg, 0.16 mmol), [(3R,4S)-4-(2,5-difluorophenyl)pyrrolidin-3-yl]carbamic acid tert-butyl ester (Preparation 48, 48 mg, 0.16 mmol) and DIPEA (29 μL, 0.18 mmol) in tert-butanol (1 mL) was heated to 80° C. until the reaction was complete The solvent was concentrated in vacuo, then purification by column chromatography (IH:EtOAc, 1:1) followed by chiral HPLC (IH:EtOH:THF 70:20:10, 15 ml/min, 290 nm, RT=11.4 min) afforded the intermediate product (R)-3-[(R)-1-(5-{2-[(3R,4S)-3-tert-butoxycarbonylamino-4-(2,5-difluorophenyl)pyrrolidin-1-yl]pyrimidin-5-yl}-[1,2,4]oxadiazol-3-yl)ethoxy]pyrrolidine-1-carboxylic acid isopropyl ester: RT=4.49 min; m/z (ES⁺)=644.5 [M+H]⁺. To a solution of the product in DCM (0.5 mL), cooled to 0° C., was added TFA (0.05 mL) and the reaction was stiffed for 2 h. A further portion of TFA (0.05 mL) was added and stirring continued until the reaction was complete. The crude mixture was passed down an SCX cartridge, eluting with MeOH then NH₄OH in MeOH (10%). The basic fraction was collected and concentrated in vacuo to afford the title compound: RT=2.83 min; ink (ES⁺)=544.0 [M+H]⁺.

### Example 26

### (S)-3-[(R)-1-(5-{2-[(3R,4S)-3-Amino-4-(2,5-difluorophenyl)pyrrolidin-1-yl]pyrimidin-5-yl}-[1,2,4]oxadiazol-3-yl)ethoxy]pyrrolidine-1-carboxylic acid isopropyl ester

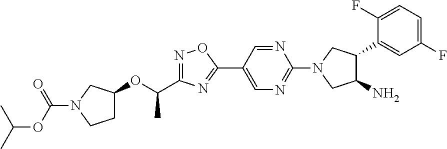

The title compound was prepared by reacting 3-{(R)-1-[5-(2-chloropyrimidin-5-yl)-[1,2,4]oxadiazol-3-yl]ethoxy}pyrrolidine-1-carboxylic acid isopropyl ester (Preparation 62) with [(3R,4S)-4-(2,5-difluorophenyl)pyrrolidin-3-yl]carbamic acid tert-butyl ester (Preparation 48) employing the procedure outlined in Example 25. Chiral HPLC: IH:EtOH:THF 70:20:10, mL/min, 290 nm, RT: 9.8 min. LCMS: RT=2.72 min; m/z (ES⁺)=544.2 [M+H]⁺.

### Example 27

### 4-[(S)-1-(5-{2-[(3S,4S)-3-Amino-4-(2-oxopiperidin-1-yl)pyrrolidin-1-yl]pyrimidin-5-yl}pyridin-2-yloxy)ethyl]piperidine-1-carboxylic acid isopropyl ester

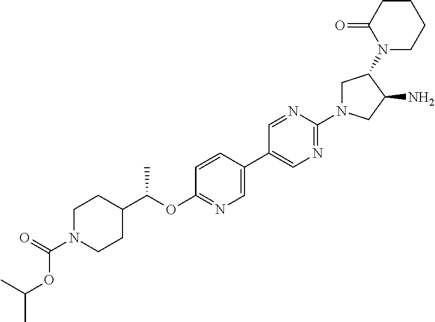

To a solution of 4-{1-[5-(4,4,5,5-tetramethyl-[1,3,2]dioxaborolan-2-yl)pyridin-2-yloxy]ethyl}piperidine-1-carboxylic acid isopropyl ester (Preparation 66, 350 mg, 0.84 mmol) and [(3S,4S)-1-(5-bromopyrimidin-2-yl)-4-(2-oxopiperidin-1-yl)pyrrolidin-3-yl]carbamic acid tert-butyl ester (Preparation 72, 442 mg, 1.00 mmol) in a combination of DMF (7 mL) and water (1.8 mL) was added [1,1-bis(diphenylphosphino)ferrocene]dichloropalladium (82 mg, 0.10 mmol) and triethylamine (418 mL, 3.00 mmol). The reaction was heated in a microwave reactor at 80° C. for 20 min, and then reacted for a further 5 min at 80° C., before being filtered through celite, washing with EtOAc. The organic mixture was washed with water, 1M citric acid, sat. NaHCO₃ solution, then brine, and dried (Na₂SO₄), before removal of the solvent in vacuo. Purification by column chromatography (IH:EtOAc, 1:1, 0:100), followed by chiral HPLC (MeCN:THF:MeOH, 67:30:3, 1 ml/min, 285 nm, RT=7.48 min), afforded the intermediate product 4-[(S)-1-(5-{2-[(3S,4S)-3-tert-butoxycarbonylamino-4-(2-oxopiperidin-1-yl)pyrrolidin-1-yl]pyrimidin-5-yl}pyridin-2-yloxy)ethyl]piperidine-1-carboxylic acid isopropyl ester: RT=4.18 min, m/z (ES⁺)=652.2 [M+H]⁺. To a solution of the product in DCM (1 mL) was added TFA (0.5 mL) and the reaction was stirred at r.t. for 1 h. The crude mixture was passed directly down an SCX cartridge, eluting with MeOH then NH₄OH in MeOH (10%). The basic fraction was collected and concentrated in vacuo to afford the title compound: RT=2.83 min, m/z (ES⁺)=552.4 [M+H]⁺.

### Example 28

### 4-[(S)-1-(5-{2-[(3R,4S)-3-Amino-4-(2,5-difluorophenyl)pyrrolidin-1-yl]pyrimidin-5-yl}pyridin-2-yloxy)ethyl]piperidine-1-carboxylic acid isopropyl ester

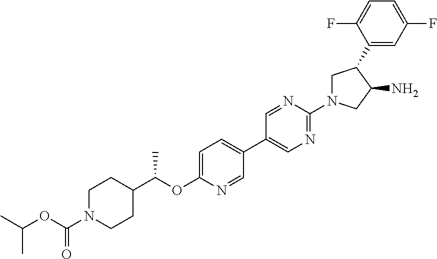

The title compound was prepared by reacting 4-{1-[5-(4,4,5,5-tetramethyl-[1,3,2]dioxaborolan-2-yl)pyridin-2-yloxy]ethyl}piperidine-1-carboxylic acid isopropyl ester (Preparation 66) with [(3R,4S)-1-(5-bromopyrimidin-2-yl)-4-(2,5-difluorophenyl)pyrrolidin-3-yl]carbamic acid tert-butyl ester (Preparation 73) employing the procedure outlined in Example 27. Chiral HPLC conditions and RT of intermediate MTBE:THF 85:15, 1 mL/min, 285 nm, RT=8.6 min. LCMS: RT=3.00 min, m/z (ES⁺)=567.3 [M+H]⁺.

### Example 29

### 4-[(R)-1-(5-{2-[(3R,4S)-3-Amino-4-(2,5-difluorophenyl)pyrrolidin-1-yl]pyrimidin-5-yl]pyridin-2-yloxy)ethyl}piperidine-1-carboxylic acid isopropyl ester

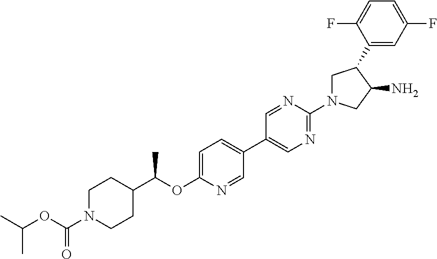

The title compound was prepared by reacting 4-{1-[5-(4,4,5,5-tetramethyl-[1,3,2]dioxaborolan-2-yl)pyridin-2-yloxy]ethyl}piperidine-1-carboxylic acid isopropyl ester (Preparation 66) with [(3R,4S)-1-(5-bromopyrimidin-2-yl)-4-(2,5-difluorophenyl)pyrrolidin-3-yl]carbamic acid tert-butyl ester (Preparation 73) employing the procedure outlined in Example 27. Chiral HPLC conditions and RT of intermediate MTBE:THF 85:15, 1 mL/min, 285 nm, RT=7.2 min. LCMS: RT=3.10 min, m/z (ES⁺)=567.3 [M+H]⁺.

The biological activity of the compounds of the invention may be tested in the following assay systems:

### GPR119 Yeast Reporter Assay

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast GPCR, Step 3 has been deleted to allow for heterologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 μg), 2 μg of each of two Fus1p-LacZ reporter plasmids (one with URA selection marker and one with TRP), 2 μg of GPR119 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 min. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μL of yeast cells added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di (β-D-galactopyranoside) was used (FDG), a substrate for the enzyme that releases fluorescein, allowing a fluorimetric read-out. 20 μL per well of 500 μM FDG/2.5% Triton X100 was added (the detergent was necessary to render the cells permeable). After incubation of the cells with the substrate for 60 min, 20 μL per well of 1M sodium carbonate was added to terminate the reaction and enhance the fluorescent signal. The plates were then read in a fluorimeter at 485/535 nm.

All of Examples 1 to 29 showed activity in this assay giving an increase in fluorescent signal of at least ˜1.5-fold that of the background signal (i.e. the signal obtained in the presence of 1% DMSO without compound). Compounds of the invention which give an increase of at least 5-fold may be preferred.

cAMP Assay

A stable cell line expressing recombinant human GPR119 was established and this cell line was used to investigate the effect of compounds of the invention on intracellular levels of cyclic AMP (cAMP). The cell monolayers were washed with phosphate buffered saline and stimulated at 37° C. for 30 min with various concentrations of compound in stimulation buffer plus 1% DMSO. Cells were then lysed and cAMP content determined using the Perkin Elmer AlphaScreen™ (Amplified Luminescent Proximity Homogeneous Assay) cAMP kit. Buffers and assay conditions were as described in the manufacturer's protocol.

Compounds of the invention produced a concentration-dependent increase in intracellular cAMP level and generally had an EC₅₀ of <10 μM. Compounds showing and EC₅₀ of less than 1 μM in the cAMP assay may be preferred.

### DPP-IV Assay Method

DPP-IV activity was measured by monitoring the cleavage of the fluorogenic peptide substrate, H-Gly-Pro-7-amino-4-methylcoumarin (GP-AMC) whereby the product 7-amino-4-methylcoumarin is quantified by fluorescence at excitation 380 nm and emission 460 nm. Assays were carried out in 96-well plates (Black OptiPlate-96F) in a total volume of 100 μL per well consisting of 50 mM Tris pH 7.6, 100 μM GP-AMC, 10-25 μU recombinant human DPP-IV and a range of inhibitor dilutions in a final concentration of 1% DMSO. Plates were read in a fluorimeter after 30 min incubation at 37° C. Recombinant human DPP-IV residues Asn29-Pro766 was purchased from BioMol.

All of Examples 1 to 53 showed activity in this assay having an IC₅₀ of <20 μM. Compounds of the invention of formula (Ia) generally have an IC₅₀ of <20 μM.

### Anti-Diabetic Effects of Compounds of the Invention in an In-Vitro Model of Pancreatic Beta Cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP Assay

HIT-T15 cells were plated in standard culture medium in 96-well plates at 100,000 cells/0.1 mL/well and cultured for 24 h and the medium was then discarded. Cells were incubated for 15 min at room temperature with 100111 stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 uL lysis buffer (5 mM HEPES, 0.3% Tween-20, 0.1% BSA, pH 7.4) was added per well and the plate was shaken at 900 rpm for 20 min. Particulate matter was removed by centrifugation at 3000 rpm for 5 min, then the samples were transferred in duplicate to 384-well plates, and processed following the Perkin Elmer AlphaScreen cAMP assay kit instructions. Briefly 25 μL reactions were set up containing 8 μL sample, 5 μL acceptor bead mix and 12 μL detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nM) to convert the readings to absolute cAMP amounts. Data was analysed using XLfit 3 software.

Representative compounds of the invention were found to increase cAMP at an EC_{K} of less than 10 μM. Compounds showing an EC₅₀ of less than 1 μM in the cAMP assay may be preferred.

### Insulin Secretion Assay

HIT-T15 cells are plated in standard culture medium in 12-well plates at 106 cells/1 ml/well and cultured for 3 days and the medium then discarded. Cells are washed ×2 with supplemented Krebs-Ringer buffer (KRB) containing 119 mM NaCl, 4.74 mM KCl, 2.54 mM CaCl₂, 1.19 mM MgSO₄, 1.19 mM KH₂PO₄, 25 mM NaHCO₃, 10 mM HEPES at pH 7.4 and 0.1% bovine serum albumin. Cells are incubated with 1 ml KRB at 37° C. for 30 min which is then discarded. This is followed by a second incubation with KRB for 30 min, which is collected and used to measure basal insulin secretion levels for each well. Compound dilutions (0, 0.1, 0.3, 1, 3, 10 μM) are then added to duplicate wells in 1 ml KRB, supplemented with 5.6 mM glucose. After 30 min incubation at 37° C. samples are removed for determination of insulin levels. Measurement of insulin was done using the Mercodia Rat insulin ELISA kit, following the manufacturers' instructions, with a standard curve of known insulin concentrations. For each well, insulin levels are corrected by subtraction of the basal secretion level from the pre-incubation in the absence of glucose. Data is analysed using XLfit 3 software.

Compounds of the invention preferably increase insulin secretion at an EC_{K} of less than 10 μM.

### Oral Glucose Tolerance Tests

The effects of compounds of the invention on oral glucose (Glc) tolerance may be evaluated in male Sprague-Dawley rats. Food is withdrawn 16 h before administration of Glc and remains withdrawn throughout the study. Rats have free access to water during the study. A cut is made to the animals' tails, then blood (1 drop) is removed for measurement of basal Glc levels 60 min before administration of the Glc load. Then, the rats are weighed and dosed orally with test compound or vehicle (20% aqueous hydroxypropyl-β-cyclodextrin) 45 min before the removal of an additional blood sample and treatment with the Glc load (2 g kg⁻¹ p.o.). Blood samples are taken from the cut tip of the tail 5, 15, 30, 60, 120, and 180 min after Glc administration. Blood glucose levels are measured just after collection using a commercially available glucose-meter (OneTouch® Ultra™ from Lifescan). Compounds of the invention preferably statistically reduce the Glc excursion at doses ≦100 mg kg⁻¹.

The effects of compounds of the invention on oral glucose (Glc) tolerance were evaluated in male C57Bl/6 or male ob/ob mice. Food was withdrawn 5 h before administration of Glc and remained withdrawn throughout the study. Mice had free access to water during the study. A cut was made to the animals' tails, then blood (20 μL) was removed for measurement of basal Glc levels 45 min before administration of the Glc load. Then, the mice were weighed and dosed orally with test compound or vehicle (20% aqueous hydroxypropyl-β-cyclodextrin or 25% aqueous Gelucire 44/14) 30 min before the removal of an additional blood sample (20 μL) and treatment with the Glc load (2-5 g kg⁻¹ p.o.). Blood samples (20 μL) were then taken 25, 50, 80, 120, and 180 min after Glc administration. The 20 μL blood samples for measurement of Glc levels were taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood were then added to 180 μL of Trinders glucose reagent (Sigma enzymatic (Trinder) colorimetric method) in a 96-well assay plate. After mixing, the samples were left at room temperature for 30 min before being read against Glc standards (Sigma glucose/urea nitrogen combined standard set). Compounds of the invention statistically reduced the Glc excursion at doses ≦100 mg kg⁻¹, for example at a dose of 30 mg kg⁻¹ the compound of Example 18 showed a >40% reduction in the Glc excursion.

1. A compound of formula (I) or a pharmaceutically acceptable salt thereof: 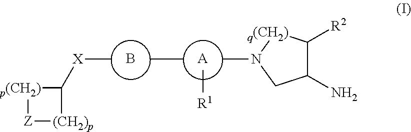
wherein p is 1 or 2;
when p is 2, Z is N—C(O)OR⁴, N—C(O)NR⁴R⁵ or N-heteroaryl optionally substituted by one or two groups selected from the group consisting of C₁₋₄ alkyl, C₃₋₆ cycloalkyl optionally substituted by C₁₋₄ alkyl, C₁₋₄ alkoxy, C₁₋₄ haloalkyl and halogen;
when p is 1, Z can also be —N—CH₂-phenyl wherein the phenyl is optionally substituted by 1 or 2 groups independently selected from the group consisting of C₁₋₄ alkyl, C₁₋₄ haloalkyl and halo;
A is a para-substituted phenyl or a para-substituted 6-membered heteroaryl ring containing one or two nitrogen atoms;
B is a 5-membered heteroaryl ring containing one of more heteroatoms selected from the group consisting of N, O and S or a para-substituted 6-membered heteroaryl ring containing one or two nitrogens;
when B is a 5-membered heteroaryl ring X is —O—CR⁶H— or —CR⁷H—O—CR⁶H—; and when B is a 6-membered heteroaryl ring X is —O— or CR⁶H—O—;
R¹ is hydrogen, halo, cyano, C₁₋₄ alkyl or C₁₋₄ haloalkyl;
q is 1 or 2;
R² is 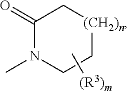
phenyl optionally substituted by one or more halo groups, or pyridyl optionally substituted by one or more halo or methyl groups;
R³ is independently halo or methyl;
n is 0 or 1;
m is 0, 1 or 2;
R⁴ is C₂₋₆ alkyl or C₃₋₆ cycloalkyl wherein the cycloalkyl is optionally substituted by C₁₋₄ alkyl;
R⁵ is hydrogen or C₁₋₄ alkyl; and
R⁶ and R² are independently hydrogen or C₁₋₂ alkyl.

2. A compound according to claim 1, or a pharmaceutically acceptable salt thereof, having the stereochemistry as defined in formula (Ia):

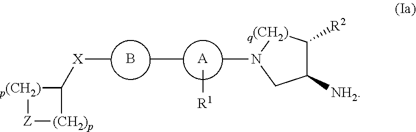

3. A compound according to claim 1, or a pharmaceutically acceptable salt thereof, wherein p is 2.

4. A compound according to claim 1, or a pharmaceutically acceptable salt thereof, wherein Z is N—C(O)OR⁴.

5. A compound according to claim 4, or a pharmaceutically acceptable salt thereof, wherein R⁴ is C₂₋₆ alkyl.

6. A compound according to claim 1, or a pharmaceutically acceptable salt thereof, wherein Z is N-heteroaryl optionally substituted by one or two groups selected from the group consisting of C₁₋₄ alkyl, C₃₋₆ cycloalkyl optionally substituted by C₁₋₄ alkyl, C₁₋₄ alkoxy, C₁₋₄ haloalkyl and halogen.

7. A compound according to claim 6, or a pharmaceutically acceptable salt thereof, wherein Z is optionally substituted oxadiazole or pyrimidine.

8. A compound according to claim 1, or a pharmaceutically acceptable salt thereof, wherein A is phenyl, pyridyl or pyrimidinyl.

9. A compound according to claim 1, or a pharmaceutically acceptable salt thereof, wherein R¹ is hydrogen.

10. A compound according to claim 1, or a pharmaceutically acceptable salt thereof, wherein B is oxadiazole, thiazole or pyridine.

11. A compound according to claim 1, or a pharmaceutically acceptable salt thereof, wherein when B is a 5-membered heteroaryl ring X is -0-CR⁶H—, and when B is a 6-membered heteroaryl ring X is CR⁶H—O—.

12. A compound according to claim 1, or a pharmaceutically acceptable salt thereof, wherein R² is phenyl substituted by one or more halo groups.

13. A compound according to claim 12, or a pharmaceutically acceptable salt thereof, wherein R² is phenyl substituted by one or more fluoro groups.

14. A compound according to claim 1, or a pharmaceutically acceptable salt thereof, wherein R⁶ and R⁷ are independently hydrogen or methyl.

15. (canceled)

16. A pharmaceutical composition comprising a compound according to claim 1, or a pharmaceutically acceptable salt thereof; and a pharmaceutically acceptable carrier.

17. A method for the treatment of a disease or condition in which GPR119 plays a role, said method comprising administering to a subject in need thereof an effective amount of a compound of claim 1, or a pharmaceutically acceptable salt thereof.

18. A method for the treatment of a disease or condition in which GPR119 and DPP-IV play a role, said method comprising administering to a subject in need thereof an effective amount of a compound according to claim 1, or a pharmaceutically acceptable salt thereof.

19. A method for the treatment of type II diabetes, said method comprising administering to a subject in need thereof an effective amount of a compound according to claim 1, or a pharmaceutically acceptable salt thereof.

20. A method for the treatment of obesity, metabolic syndrome (syndrome X), impaired glucose tolerance, hyperlipidemia, hypertriglyceridemia, hypercholesterolemia, low HDL levels or hypertension, said method comprising administering to a patient in need thereof an effective amount of a compound according to claim 1, or a pharmaceutically acceptable salt thereof.

21. A compound according to claim 1 which is selected from the group consisting of:
4-(5-{6-[(3R,4S)-3-Amino-4-(2-fluorophenyl)pyrrolidin-1-yl]pyridine-3-yl}-[1,2,4]oxadiazol-3-ylmethoxy)piperidine-1-carboxylic acid isopropyl ester or a pharmaceutically acceptable salt thereof,
4-(5-{2-[(3R,4S)-3-Amino-4-(2-fluorophenyl)pyrrolidin-1-yl]pyrimidin-5-yl}-[1,2,4]oxadiazol-3-ylmethoxy)piperidine-1-carboxylic acid isopropyl ester p-toluenesulfonic acid salt or a free base thereof,
4-(5-{2-[(3R,4S)-3-Amino-4-(2,5-difluorophenyl)pyrrolidin-1-yl]pyrimidin-5-yl}-[1,2,4]oxadiazol-3-ylmethoxy)piperidine-1-carboxylic acid isopropyl ester p-toluenesulfonic acid salt or a free base thereof,
4-(5-{2-[(3R,4S)-3-Amino-4-(2,4-difluorophenyl)pyrrolidin-1-yl]pyrimidin-5-yl}-[1,2,4]oxadiazol-3-ylmethoxy)piperidine-1-carboxylic acid isopropyl ester p-toluenesulfonic acid salt or a free base thereof,
4-(3-{6-[(3R,4S)-3-Amino-4-(2-fluorophenyl)pyrrolidin-1-yl]pyridine-3-yl}-[1,2,4]oxadiazol-5-ylmethoxy)piperidine-1-carboxylic acid isopropyl ester p-toluenesulfonic acid salt or a free base thereof,
4-(3-{6-[(3S,4R)-3-Amino-4-(2-fluorophenyl)pyrrolidin-1-yl]pyridine-3-yl}-[1,2,4]oxadiazol-5-ylmethoxy)piperidine-1-carboxylic acid isopropyl ester p-toluenesulfonic acid salt or a free base thereof,
4-(3-{6-[(3S,4R)-3-Amino-4-(2-fluorophenyl)pyrrolidin-1-yl]pyridine-3-yl}-[1,2,4]oxadiazol-5-ylmethoxy)piperidine-1-carboxylic acid isopropyl ester p-toluenesulfonic acid salt or a free base thereof,
4-(3-{6-[(3R,4S)-3-Amino-4-(2,5-difluorophenyl)pyrrolidin-1-yl]pyridin-3-yl}-[1,2,4]oxadiazol-5-ylmethoxy)piperidine-1-carboxylic acid isopropyl ester p-toluenesulfonic acid salt or a free base thereof,
4-[(R)-1-(3-{6-[(3R,4S)-3-Amino-4-(2,5-difluorophenyl)pyrrolidin-1-yl]pyridin-3-yl}-[1,2,4]oxadiazol-5-yl)ethoxy]piperidine-1-carboxylic acid isopropyl ester p-toluenesulfonic acid salt or a free base thereof,
4-[(R)-1-(5-{2-[(3R,4S)-3-Amino-4-(2,5-difluorophenyl)pyrrolidin-1-yl]pyrimidin-5-yl}-[1,2,4]oxadiazol-3-yl)ethoxy]piperidine-1-carboxylic acid isopropyl ester or a pharmaceutically acceptable salt thereof,
4-[(R)-1-(5-{5-[(3R,4S)-3-Amino-4-(2,5-difluorophenyl)pyrrolidin-1-yl]pyrazin-2-yl}-[1,2,4]oxadiazol-3-yl)ethoxy]piperidine-1-carboxylic acid isopropyl ester or a pharmaceutically acceptable salt thereof,
4-{5-[(3S,4R)-3-Amino-4-(2,5-difluorophenyl)-3,4,5,6-tetrahydro-2H-[1,2]bipyridinyl-5′yl]-[1,2,4]oxadiazol-3-ylmethoxy}piperidine-1-carboxylic acid isopropyl ester hydrochloride or a free base thereof,
4-{3-[(3R,4R)-3-Amino-4-(2,5-difluorophenyl)-3,4,5,6-tetrahydro-2H-[1,2]bipyridinyl-5′-yl]-[1,2,4]oxadiazol-5-ylmethoxy}piperidine-1-carboxylic acid isopropyl ester dihydrochloride or a free base thereof,
4-(3-{2-[(3R,4S)-3-Amino-4-(2,4-difluorophenyl)pyrrolidin-1-yl]pyrimidin-5-yl}-[1,2,4]oxadiazol-5-ylmethoxy)piperidine-1-carboxylic acid isopropyl ester p-toluenesulfonic acid salt or a free base thereof,
4-(3-{2-[(3R,4S)-3-Amino-4-(2,5-difluorophenyl)piperidin-1-yl]pyrimidin-5-yl}-[1,2,4]oxadiazol-5-ylmethoxy)piperidine-1-carboxylic acid isopropyl ester p-toluenesulfonic acid salt or a free base thereof,
4-[(R)-1-(3-{2-[(3R,4S)-3-Amino-4-(2,4-difluorophenyl)pyrrolidin-1-yl]pyrimidin-5-yl}-[1,2,4]oxadiazol-5-yl)ethoxy]piperidine-1-carboxylic acid isopropyl ester p-toluenesulfonic acid salt or a free base thereof,
4-[(R)-1-(3-{2-[(3R,4S)-3-Amino-4-(2,5-difluorophenyl)pyrrolidin-1-yl]pyrimidin-5-yl}-[1,2,4]oxadiazol-5-yl)ethoxy]piperidine-1-carboxylic acid isopropyl ester p-toluenesulfonic acid salt or a free base thereof,
4-(3-{2-[(3R,4S)-3-Amino-4-(2-fluorophenyl)pyrrolidin-1-yl]pyrimidin-5-yl}-[1,2,4]oxadiazol-5-ylmethoxy)piperidine-1-carboxylic acid isopropyl ester p-toluenesulfonic acid salt or a free base thereof,
4-[(R)-1-(3-{2-[(3R,4S)-3-Amino-4-(2-fluorophenyl)pyrrolidin-1-yl]pyrimidin-5-yl}-[1,2,4]oxadiazol-5-yl)ethoxy]piperidine-1-carboxylic acid isopropyl ester p-toluenesulfonic acid salt or a free base thereof,
4-[(R)-1-(5-{6-[(3R,4S)-3-Amino-4-(2,5-difluorophenyl)pyrrolidin-1-yl]pyridazin-3-yl}-[1,2,4]oxadiazol-3-yl)ethoxy]piperidine-1-carboxylic acid isopropyl ester or a pharmaceutically acceptable salt thereof,
3-[(R)-1-(3-{2-[(3R,4S)-3-Amino-4-(2,5-difluorophenyl)pyrrolidin-1-yl]pyrimidin-5-yl}-[1,2,4]oxadiazol-5-yl)ethoxy]azetidine-1-carboxylic acid isopropyl ester p-toluenesulfonic acid salt or a free base thereof,
4-[(R)-1-(3-{5-[(3R,4S)-3-Amino-4-(2,5-difluorophenyl)pyrrolidin-1-yl]pyrazin-2-yl}-[1,2,4]oxadiazol-5-yl)ethoxy]piperidine-1-carboxylic acid isopropyl ester or a pharmaceutically acceptable salt thereof,
4-[(R)-1-(5-{2-[(3R,4S)-3-Amino-4-(2,4,5-trifluorophenyl)pyrrolidin-1-yl]pyrimidin-5-yl}-[1,2,4]oxadiazol-3-yl)ethoxy]piperidine-1-carboxylic acid isopropyl ester or a pharmaceutically acceptable salt thereof,
(R)-3-[(R)-1-(5-{2-[(3R,4S)-3-Amino-4-(2,5-difluorophenyl)pyrrolidin-1-yl]pyrimidin-5-yl}-[1,2,4]oxadiazol-3-yl)ethoxy]pyrrolidine-1-carboxylic acid isopropyl ester or a pharmaceutically acceptable salt thereof,
(S)-3-[(R)-1-(5-{2-[(3R,4S)-3-Amino-4-(2,5-difluorophenyl)pyrrolidin-1-yl]pyrimidin-5-yl}-[1,2,4]oxadiazol-3-yl)ethoxy]pyrrolidine-1-carboxylic acid isopropyl ester or a pharmaceutically acceptable salt thereof,
4-[(S)-1-(5-{2-[(3S,4S)-3-Amino-4-(2-oxopiperidin-1-yl)pyrrolidin-1-yl]pyrimidin-5-yl}pyridin-2-yloxy)ethyl]piperidine-1-carboxylic acid isopropyl ester or a pharmaceutically acceptable salt thereof,
4-[(S)-1-(5-{2-[(3R,4S)-3-Amino-4-(2,5-difluorophenyl)pyrrolidin-1-yl]pyrimidin-5-yl}pyridin-2-yloxy)ethyl]piperidine-1-carboxylic acid isopropyl ester or a pharmaceutically acceptable salt thereof, and
4-[(R)-1-(5-{2-[(3R,4S)-3-Amino-4-(2,5-difluorophenyl)pyrrolidin-1-yl]pyrimidin-5-yl}pyridin-2-yloxy)ethyl]piperidine-1-carboxylic acid isopropyl ester or a pharmaceutically acceptable salt thereof.
